(19) United States
(12) Patent Application Publication
(10) Pub. No.: US 2004/0015154 A1
(43) Pub. Date: Jan. 22, 2004

(54) Implantable devices with invasive and non-invasive reversible infusion rate adjustability

(75) Inventors: Derek J. Harper, Goleta, CA (US); Charles F. Milo, Mountain View, CA (US)

Correspondence Address: YOUNG LAW FIRM, A PROFESSIONAL CORPORATION, 4370 ALPINE ROAD SUITE 106, PORTOLA VALLEY, CA, 94028

(73) Assignee: MicroSolutions, Inc., Goleta, CA

(21) Appl. No.: 10/386,919

(22) Filed: Mar. 11, 2003

Related U.S. Application Data
(63) Continuation-in-part of application No. 09/838,662, filed on Apr. 19, 2001, now Pat. No. 6,632,217.
(60) Provisional application No. 60/363,599, filed on Mar. 12, 2002.
(60) Provisional application No. 60/396,831, filed on Jul. 16, 2002.

(51) Int. Cl. A61K 9/22

(52) U.S. Cl. 604/892.1

(57) ABSTRACT

An implantable pump for delivering a pharmaceutical agent includes a pump engine, a piston, a pharmaceutical agent compartment and a rate adjustment assembly. The pharmaceutical agent compartment is configured to enclose a volume of pharmaceutical agent and the piston. When the piston is acted upon by the pump engine, the piston moves within the pharmaceutical agent compartment along a substantially circular path and delivers the pharmaceutical agent. The rate adjustment assembly is configured to enable a selective and reversible increase or decrease of the delivery rate of the pharmaceutical agent.

### CROSS-REFERENCE TO RELATED APPLICATIONS

This application is a Continuation-In-Part (CIP) of U.S. patent application Ser. No. 09/838,662 filed on Apr. 19, 2001, attorney docket number MICR5701, and claims priority to U.S. provisional application No. 60/363,599 filed on Mar. 12, 2002, Attorney docket number MICR5790 and also claims priority to U.S. provisional application No. 60/396,831 filed on Jul. 16, 2002, attorney docket number MICR5820, the disclosures of which are incorporated herewith in their entirety.

### BACKGROUND OF THE INVENTION

1. Field of the Invention

The claimed invention relates generally to the field of drug delivery systems. In particular, the claimed invention relates to implantable pump systems that include an upward and downward infusion rate adjustability functionality.

2. Description of the Related Art

Since the beginning of modem medicine, drugs have been administered orally. Patients have taken pills as recommended by their physician. The pills must pass through the digestive system and then the liver before they reach their intended delivery site (e.g., the vascular system). The actions of the digestive tract and the liver typically reduce the efficacy of medication by about 33%. Furthermore, oral medications must be administered by the patient. Patient compliance to the prescribed delivery profile is often poor. Studies suggest that 40% of patients do not comply with their oral medication consumption instructions. This causes two concerns. First, patients who do not take their medication as instructed are not maintaining blood drug levels within the therapeutic window and are therefore not receiving adequate therapy for their disease. A second, worse scenario than receiving too little medication occurs when the patient may be taking too much medication either by accident or purposefully in order to make up for a missed dose. Both of these patient-controlled scenarios can be dangerous to the patient, and at a minimum may prolong or aggravate their disease. Subcutaneous drug delivery and intravenous drug delivery have the advantage of bypassing the acidic and enzymatic action of the digestive system. Unfortunately, IV administration requires the use of a percutaneous catheter or needle to deliver the drug to the vein. The percutaneous site requires extra cleanliness and maintenance to minimize the risk of infection. Infection is such a significant risk that IV administration is often limited to a number of weeks, at most. In addition, the patient must wear an external pump connected to the percutaneous catheter if the therapy is intended to last longer than a few hours and the patient desires to be ambulatory. Subcutaneous drug delivery can be either partially implanted or totally implanted. Partially implanted systems rely on a percutaneous catheter or needle stick to deliver the medication, therefore, partially implanted systems have the same limitations as IV systems. Totally implanted systems have fewer maintenance requirements and are far less prone to infection than IV or partially implanted systems.

In the 1970s, a new approach toward sustained drug delivery was commercialized for animal use only. The driving force of such pumps was based upon a new approach utilizing the principle of osmosis. A recent example of such a pump is described listed in U.S. Pat. No. 5,728,396. This patent discloses an implantable osmotic pump that achieves a sustained delivery of leuprolide. The pump includes a right-cylindrical impermeable reservoir that is divided into a water-swellable agent chamber and a drug chamber, the two chambers being divided by a movable piston. Fluid from the body is imbibed through a semipermeable membrane into the water-swellable agent chamber. As the water-swellable agent in the water-swellable agent chamber expands in volume, it pushes on the movable piston, which correspondingly decreases the volume of the drug chamber and causes the drug to be released through a diffusion outlet at a substantially constant rate.

A limitation of the osmotic pump disclosed in the above-identified patent, however, is that its infusion rate cannot be adjusted once it is implanted. This is acceptable for medications that do not need rate adjustment, but often physicians desire to adjust the infusion rate based on the clinical status of the patient. One example of when a physician would want to increase the infusion rate is in the field of pain management. Osmotic pumps can be used to deliver medication to treat pain lasting over an extended period of time. Pain, however, often increases with time, and sometimes patients become tolerant to pain medications; therefore, more medication is needed to effectively treat the pain. The system disclosed in the above-identified patent does not allow a rate increase or decrease (other than after the available drug supply has been exhausted) after implantation, so the physician must surgically remove the current implant and implant an additional pump to deliver the correct dosage. However, the prospect of yet another surgical procedure may cause many patients to forego the potential benefits of the larger dose and may also cause their physicians to advise against the initial procedure altogether. In some cases, it may also be advisable to decrease the dose of pharmaceutical agent delivered by the implantable pump without removing the pump from the patient and without breaching the patient's skin.

The aspect ratio of conventional cylindrical osmotic pump delivery devices is large, and often not compatible with the human body. Indeed, the human body does not have naturally-formed right-cylindrical cavities in which to implant such devices in the patient, in an unobtrusive and comfortable manner.

What are needed, therefore, are improved osmotic pumps. What are also needed are improved implantable osmotic pumps that conform to the patient's anatomy and that more closely match the topology of the implant site. Also needed are novel implantable osmotic pumps for long term delivery of a pharmaceutical agent that do not rely upon a right-cylindrical pharmaceutical agent compartment and/or conventional cylindrical pistons. Also needed are implantable pumps that enable the physician to increase or decrease the dose of pharmaceutical agent delivered to the patient without, however, removing the pump from the implant site. Moreover, there is also a need for implantable pumps whose infusion rates are freely adjustable, up or down (and back again, if needed). While it may sometimes be acceptable to breach the patient's skin to effectuate such infusion rate adjustments, it may be preferable to have the ability to make up and down infusion rate adjustments that do not require the physician or caregiver to breach the patient's skin to make the required or desired infusion rate adjustment on an previously implanted pump. Also desirable is an implantable pump that includes an adjustment mechanism that allows the physician to select an “off” position where the pump does not infuse any medication.

### SUMMARY OF THE INVENTION

According to an embodiment thereof, the present invention is a pump for delivering a pharmaceutical agent, comprising a pump engine; a piston; a pharmaceutical agent compartment configured to enclose a volume of pharmaceutical agent and the piston, the pharmaceutical agent compartment being configured such that when the piston is acted upon by the pump engine, the piston moves within the pharmaceutical agent compartment along a substantially circular path and delivers the pharmaceutical agent, and a rate adjustment assembly configured to enable a selective and reversible increase or decrease of a delivery rate of the pharmaceutical agent.

The rate adjustment assembly may be configured to selectively vary the delivery rate of the pharmaceutical agent by percutaneous insertion and manipulation of a rate adjustment tool in the rate adjustment assembly. The rate adjustment assembly may be configured to vary the delivery rate of the pharmaceutical agent non-invasively when the pump is implanted into a patient. The rate adjustment module may be configured to enable the delivery rate of the pharmaceutical agent to be changed by application of an external magnetic field to the pump. The pharmaceutical agent compartment may be preloaded with a volume of pharmaceutical agent.

According to another embodiment, the present invention may be viewed as a method of delivering a pharmaceutical agent, comprising steps of: implanting a pump into the patient, the pump including a pump engine, a piston, a pharmaceutical agent compartment configured to enclose a volume of pharmaceutical agent and the piston, the pharmaceutical agent compartment being configured such that when the piston is acted upon by the pump engine, the piston moves within the pharmaceutical agent compartment along a substantially circular path and delivers the pharmaceutical agent, and a rate adjustment assembly configured to enable a selective and reversible increase or decrease of a delivery rate of the pharmaceutical agent, and manipulating the rate adjustment assembly to selectively increase or decrease the delivery rate of the pharmaceutical agent.

The implanting step may include a step of making an incision in the patient near a desired implantation site and the manipulating step may be carried out after the implantation step and after the incision is closed. The manipulation step may include a step of percutaneously inserting a rate adjustment tool into the rate adjustment assembly. The manipulation step may be carried out without breaching the patient's skin. The manipulation step may include a step of applying an external magnetic field near the implantation site. The external magnetic field applying step may include a step of rotating the external magnetic field by a selected degree of rotation.

The present invention, according to another embodiment thereof, is an osmotic pump, comprising: an osmotic engine; a pump housing enclosing the osmotic engine and defining a substantially toroidal space adapted to contain a volume of pharmaceutical agent, and a rate adjustment module configured to enable a selective and reversible increase or decrease of a delivery rate of the pharmaceutical agent. The osmotic pump may be preloaded with a volume of pharmaceutical agent.

According to still another embodiment, the present invention is an osmotic pump for delivery a pharmaceutical agent, comprising: an osmotic engine; a pharmaceutical agent compartment adapted to contain a volume of the pharmaceutical agent; a plurality of semipermeable membranes, one end of each of which being in communication with the osmotic engine, each of the plurality of semipermeable membranes being configured to enable an osmotic pressure differential to develop when another end thereof is selectively exposed to fluid from an environment of use, and a rate adjustment assembly configured to selectively expose or cover at least one of the plurality of semipermeable membranes to the environment of use to selectively and reversibly increase or decrease a rate at which the pharmaceutical agent is delivered from the osmotic pump.

The rate adjustment module may be configured to enable the selective and reversible increase or decrease of the delivery rate without physical contact with the pump. The rate adjustment module may be configured to enable the selective and reversible increase or decrease of the delivery rate through an application of an external magnetic field to the osmotic pump. The rate adjustment assembly may be further configured to mate with a rate adjustment tool. The osmotic pump may be preloaded with the volume of the pharmaceutical agent.

The present invention is also, according to yet another embodiment thereof, a method of non-invasively increasing or decreasing a dose of pharmaceutical agent delivered to a patient by a previously implanted osmotic pump, comprising the steps of: providing a magnet; positioning the provided magnet on or close to a skin of the patient over the previously implanted osmotic pump, and rotating the positioned magnet by a predetermined degree of rotation, whereby the implanted osmotic pump responds to the rotating magnet by increasing or decreasing the dose of pharmaceutical agent delivered to the patient.

### BRIEF DESCRIPTION OF THE DRAWINGS

For a further understanding of the objects and advantages of the claimed invention, reference should be made to the following detailed description, taken in conjunction with the accompanying figures, in which:

FIG. 1 is a perspective view of the osmotic pump according to an embodiment of the present invention.

FIG. 2 is an exploded view of the osmotic pump according to an embodiment of the present invention, showing the major components thereof.

FIG. 3 is a plan view of the osmotic pump according to an embodiment of the present invention in which the first half of the housing has been removed.

FIG. 4 is a cross sectional view of the osmotic pump of FIG. 3, taken along lines BB′.

FIG. 5 is a cross sectional view of the osmotic pump of FIG. 3, taken along lines AA′.

FIG. 6 is a plan view of the second half of the osmotic pump housing, according to an embodiment of the present invention.

FIG. 7 is a cross sectional view of the second half of the osmotic pump housing, taken along lines CC′.

FIG. 8 is a perspective view of the first half of the osmotic pump housing according to an embodiment of the present invention.

FIG. 9 is a plan view of the first half of the osmotic pump housing of FIG. 8.

FIG. 10 is a cross-sectional view of the first half of the osmotic pump housing of FIG. 9, taken along lines DD′.

FIG. 11 is a plan view of an embodiment of the membrane enclosure, according to an embodiment thereof.

FIG. 12 is a perspective view of the membrane enclosure of FIG. 11, showing the semipermeable membrane wells in dashed lines.

FIG. 13 is a plan view of an impermeable membrane can of an osmotic pump according to an embodiment of the present invention, showing the internal surface and through bore thereof in dashed lines.

FIG. 14 shows a side view of the impermeable membrane can of FIG. 13.

FIG. 15 is a plan view of the osmotic engine of the osmotic pump, according to an embodiment of the present invention.

FIG. 16 is a side view of the osmotic engine of FIG. 15.

FIG. 17 is a plan view of the coiled tube, according to an embodiment of the present invention.

FIG. 18 is a cross-sectional view of the tube of FIG. 17, taken along line EE′.

FIG. 19 is a cross-sectional view of the coiled tube of FIG. 17, taken along line FF′.

FIG. 20 illustrates the tube coupled to a catheter, according to an embodiment of the present invention.

FIG. 21 illustrates the distal tip of the catheter of FIG. 20, according to an embodiment of the present invention.

FIG. 22 illustrates the proximal end of the catheter of FIG. 20, according to an embodiment of the present invention.

FIG. 23 shows an embodiment of a piston within the coiled pharmaceutical agent compartment, according to an embodiment of the present invention.

FIG. 24 shows a further embodiment of a piston within the coiled pharmaceutical agent compartment, according to an embodiment of the present invention.

FIG. 25 shows a further embodiment of still another piston within the coiled pharmaceutical agent compartment, according to an embodiment of the present invention.

FIG. 26 shows a first step of a method by which the impermeable membrane of the first impermeable membrane may be breached so as to escalate a dose of pharmaceutical agent delivered to the patient, according to an embodiment of the present invention.

FIG. 27 shows a second step of a method by which the impermeable membrane of the first impermeable membrane may be breached so as to escalate a dose of pharmaceutical agent delivered to the patient, according to an embodiment of the present invention.

FIG. 28 shows a third step of a method by which the impermeable membrane of the first impermeable membrane can may be breached so as to escalate a dose of pharmaceutical agent delivered to the patient, according to an embodiment of the present invention.

FIG. 29 shows a fourth step of a method by which the impermeable membrane of the second impermeable membrane can may be breached so as to further escalate a dose of pharmaceutical agent delivered to the patient, according to an embodiment of the present invention.

FIG. 30 shows a fifth step of a method by which the impermeable membrane of the second impermeable membrane can may be breached so as to further escalate a dose of pharmaceutical agent delivered to the patient, according to an embodiment of the present invention.

FIG. 31 shows a sixth step of a method by which the impermeable membrane of the second impermeable membrane can may be breached so as to further escalate a dose of pharmaceutical agent delivered to the patient, according to an embodiment of the present invention.

FIG. 32 is a plan view of another embodiment of the membrane enclosure, according to the present invention, showing the OFF feature of the present invention.

FIG. 33 is a perspective view of the membrane enclosure of FIG. 32, showing the semipermeable membrane wells in dashed lines and the OFF switch feature of an embodiment of the present invention.

FIG. 34 is an exploded view of another embodiment of an osmotic pump according to an embodiment of the present invention.

FIG. 35 is an exploded view of a three-stage osmotic pump, according to another embodiment of the present invention.

FIG. 36A is a top view of a three stage osmotic pump according to an embodiment of the present invention, showing the internal structure thereof in dashed lines.

FIG. 36B is a reduced-size (relative to FIG. 36a) top view of a three stage osmotic pump, showing selected exemplary dimensions thereof.

FIG. 37 is a cross-sectional view of a three stage osmotic pump according to an embodiment of the present invention, taken along cross-sectional line BB′ of FIG. 36.

FIG. 38 is a cross-sectional view of a three stage osmotic pump according to an embodiment of the present invention, taken along cross-sectional line AA′ of FIG. 36.

FIG. 39 is a cross-sectional view of the filter assembly 312 of FIG. 35.

FIG. 40 is a front view of the filter assembly 312 of FIG. 35.

FIG. 41 is a cross-sectional view of a piston, according to an embodiment of the present invention.

FIG. 42 is a perspective view of a single stage osmotic pump according to another embodiment of the present invention.

FIG. 43 is an exploded view of a single stage osmotic pump according to an embodiment of the present invention.

FIG. 44 is a top view of a single stage osmotic pump according to an embodiment of the present invention, showing internal components thereof in dashed lines.

FIG. 45 is an exploded view of an osmotic pump with forward and backward reversible rate adjustability features, according to an embodiment of the present invention.

FIG. 46 is a top line drawing view of the pump of FIG. 45, showing additional structure thereof.

FIG. 47 is a cross-sectional view of the pump of FIG. 46, taken along cross-sectional line I-I.

FIG. 48 shows an isometric line drawing view of an osmotic pump with forward and backward rate adjustability features, shown with an exemplary rate adjustment tool, according to an embodiment of the present invention.

FIG. 49 is a partially exploded view of the pump of FIG. 48, to show additional structure of the rate adjustment assembly thereof, according to an embodiment of the present invention.

FIG. 50 shows a sectioned isometric view of an osmotic pump with non-invasive, upward and downward reversible infusion rate adjustability, according to another embodiment of the present invention.

FIG. 51 is a top line drawing view of an osmotic pump with non-invasive, upward and downward reversible infusion rate adjustability, according to an embodiment of the present invention.

FIG. 52 is a cross-sectional view of the osmotic pump of FIG. 51, taken along cross-sectional line I-I.

FIG. 53 is an isometric view of an central rate adjustment module of an implantable osmotic pump with upward and downward reversible infusion rate adjustability, according to an embodiment of the present invention.

FIG. 54 is a top view of the central rate adjustment module of FIG. 53.

FIG. 55 is a cross-sectional view of the central rate adjustment module shown in FIG. 54, taken along cross-sectional line I-I thereof.

FIG. 56 is an isometric view of a magnet sleeve of an implantable osmotic pump with non-invasive, upward and downward reversible infusion rate adjustability, according to an embodiment of the present invention.

FIG. 57 is a plan view of the magnet sleeve of FIG. 56.

FIG. 58 is a cross-sectional view of the magnet sleeve of FIG. 57, taken along cross-sectional line I-I thereof.

### DESCRIPTION OF THE INVENTION

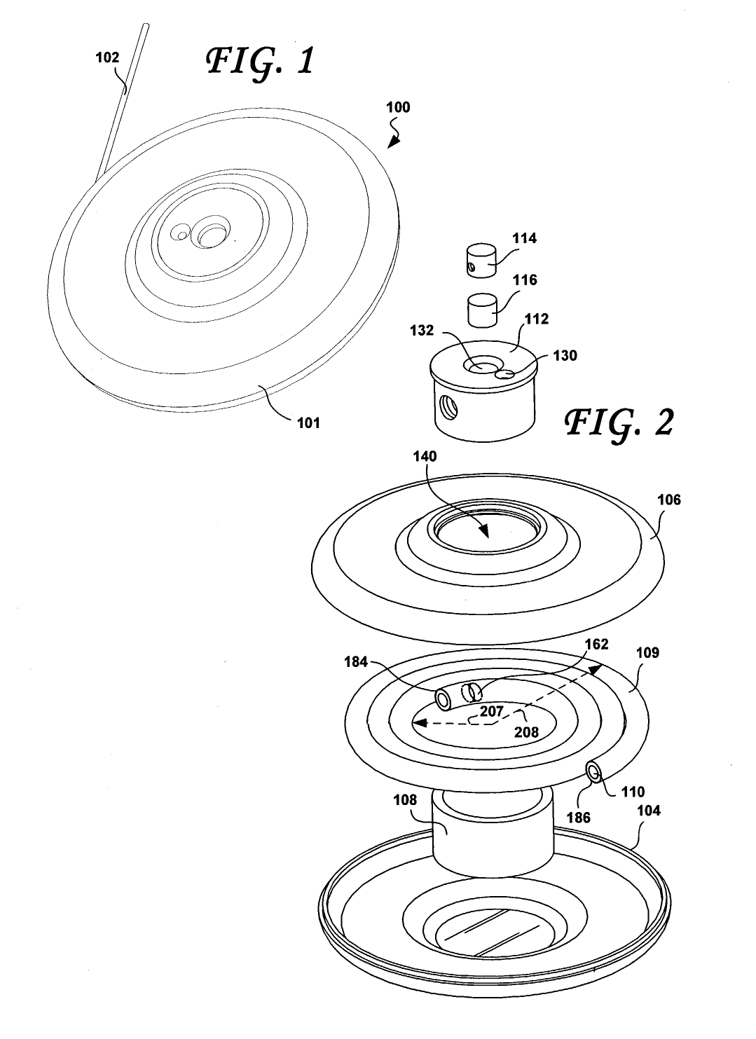

FIG. 1 is a perspective view and FIG. 2 shows an exploded view of the pump 100 according to an embodiment of the present invention. Considering FIGS. 1 and 2 collectively, the pump 100 includes a pump engine 108 and a substantially toroidal compartment around the engine 108. The toroidal compartment is bounded by an inner radius 207 and an outer radius 208 and is adapted to contain a fluid, such as a pharmaceutical agent. According to an embodiment of the present invention, the pharmaceutical agent compartment is tube-shaped and is defined by an inner lumen 110 of a tube 109 that may be coiled at least partially around the osmotic engine 108. The tube 109 has a proximal end 184 and a distal end 186. The tube 109 may include or be formed of, for example, polyimid. A piston 162 is disposed in the tube-shaped compartment 110. The piston is adapted to travel (in the direction from the proximal end 184 to the distal end 186 of the tube 109) within the tube-shaped compartment 110 and to cause a volume of fluid to be forced out of the distal end 186 of the tube 109. As shown in FIG. 1, a catheter 102 may be coupled to the distal end 186 of the tube 109, to enable the fluid forced out the distal end 186 of the tube 109 to be delivered to the intended delivery site within the patient. In one embodiment of the present invention, the pump engine 108 includes an osmotic engine. The pump 100 may further include a pump housing 101 that is configured to enclose (at least) the pump engine 108 and the tube 109. As shown in FIG. 2, the pump housing 101 may include a first housing half 106 and a mating second housing half 104. According to an embodiment of the present invention, the first and second pump housing halves 106, 104 mate to one another like a clamshell, in a fluid-tight fashion. As shown, the first and second housing halves 106, 104 may each have a generally circular outline (as may the entire pump 100) and have a generally define a saucer shape. The first housing half 106 may further define an opening 140, which may be circular in shape.

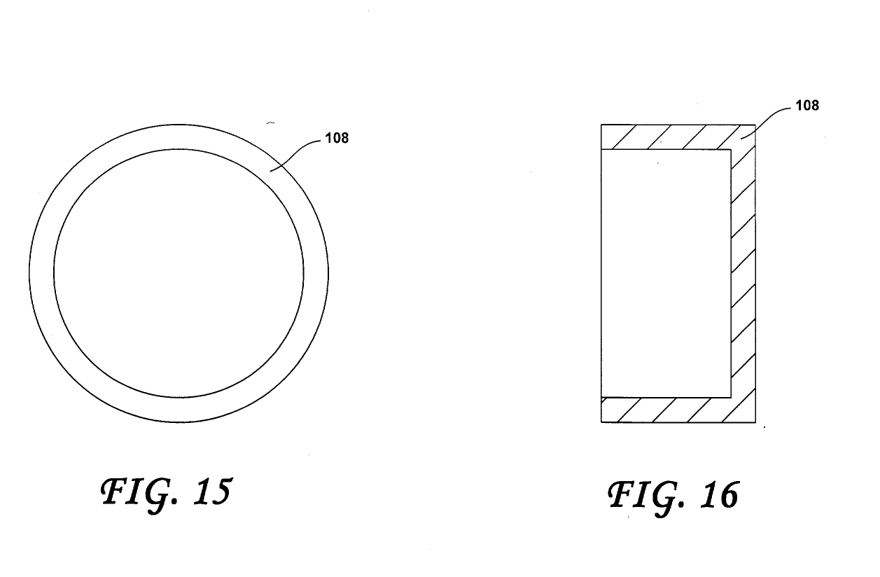

Embodiments of the present invention will now be described in terms of an implantable osmotic pump for delivering a pharmaceutical agent to a patient, although the claimed inventions are not so limited. The pump and/or the catheter 102 may be implanted intravascularly, subcutaneously, epidurally, intrathecally and/or intraventricularly, for example. As shown in FIG. 2 as well as in FIGS. 15 and 16, the pump engine 108 (referred to hereafter as osmotic engine 108, although the claimed inventions are not limited to osmotic-type pump engines) may be shaped like hollow, open-ended right cylinder. The osmotic engine 108 is hygroscopic and may include a salt block or a “salt wafer” and/or may include an absorbent polymer, such as poly(acrylic acid), potassium salt; poly(acrylic acid), sodium salt; poly(acrylic acid-co-acrylamide), potassium salt; poly(acrylic acid), sodium salt-graft-poly(ethylene oxide); poly(2-hydroxethyl methacrylate) and/or poly(2-hydroxypropyl methacrylate) and poly(isobutylene-co-maleic acid). Suitable absorbent polymers are available from Aldrich, Inc. of Milwaukee, Wis., for example. The osmotic engine 108 may include a base that may be disposed in a correspondingly shaped depression defined in the second housing half 104 and a cylindrical wall attached to the base.

According to an embodiment of the present invention, the pump 100 may include a generally cylindrical-shaped membrane enclosure 112. The membrane enclosure 112 may be fitted within and partially surrounded by the pump engine 108. The membrane enclosure 112 is dimensioned to closely fit the opening 140 defined in the first housing half 106. The membrane enclosure 112 may include an initial dose semipermeable membrane (formed of or including cellulose acetate, for example), as shown in FIG. 5, to create a fluid path for water through the initial water access port 130 defined in the membrane enclosure 112 to the osmotic engine 108. The initial water access port 130 may be spanned by a thin impermeable membrane 182, thereby defining an interstitial space between the initial dose semipermeable membrane and the impermeable membrane. This interstitial space may be filled with a saturated saline solution, to keep the initial dose semipermeable membrane fully hydrated prior to implantation of the pump 100 in a patient (not shown). Prior to implantation, the physician may breach the impermeable membrane 182 spanning the initial water access port 130 to allow water from the patient to enter the initial dose semipermeable membrane well 150 (see FIG. 12) and migrate across the initial dose semipermeable membrane 134 (see FIG. 5) to reach the osmotic engine 108. In this manner, the initial water access port 130, the thin impermeable membrane 182 and the saturated saline solution effectively form a pump ON switch. Indeed, after implantation of the pump but before breaching the thin impermeable membrane 182, the pump 100 does not deliver any pharmaceutical agent to the patient. It is only after breaching the thin impermeable membrane 182 that the pump becomes effective to initiate delivery of the contained pharmaceutical agent to the patient. The saturated saline solution between the impermeable membrane 182 and the underlying initial dose semipermeable membrane 150 insures that the onset of delivery of the pharmaceutical agent is not delayed by the time required for the initial dose semipermeable membrane 150 to hydrate.

The membrane enclosure 112 may also define a primary water access port 132 that may be (but need not be) concentric with the circumference of the membrane enclosure 112. A dose escalation assembly may fit within the primary water access port 132. The dose escalation assembly, according to an embodiment of the present invention, is adapted to selectively increase the amount of water from implantation site within the patient that reaches the osmotic engine 108. The dose escalation assembly may include one or more impermeable membrane cans fitted within the primary water access port 132 of the membrane enclosure 112. In the embodiment of FIG. 2, the dose escalation includes a first impermeable membrane can 114 stacked upon a second impermeable membrane can 116 whose structure and function is described hereunder.

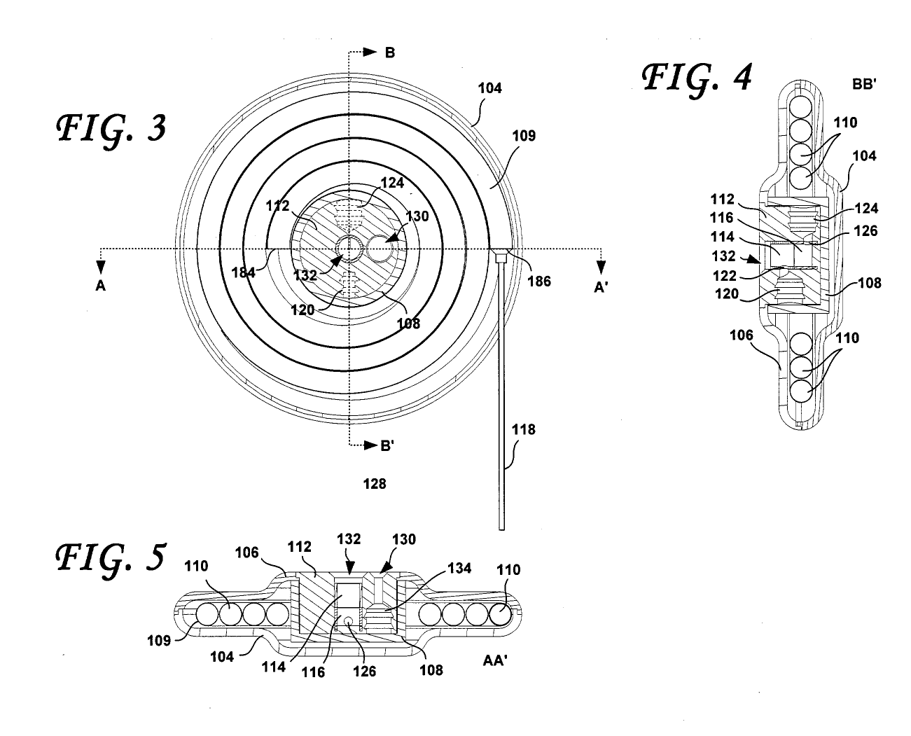

Reference is now made to FIGS. 3-5, in which FIG. 3 is a plan view of the osmotic pump according to an embodiment of the present invention in which the first half of the housing has been removed, FIG. 4 is a cross sectional view of the osmotic pump of FIG. 3, taken along lines BB′ of FIG. 3 and FIG. 5 is a cross sectional view of the osmotic pump of FIG. 3, taken along lines AA′. FIG. 3 shows the tube 109 coiled around the osmotic engine 108 from the proximal end 184 to the distal end thereof, shown at 186. The distal end 186 of the coiled tube 109 may be fitted with a catheter ID tube 118 that facilitates the coupling of the catheter 102 to the distal end 186 of the tube 109. As shown in FIG. 5, the initial water access port 130 may lead to an initial dose semipermeable membrane 134 within the membrane enclosure 112. The membrane enclosure 112 is configured to enable water from the patient to flow into the initial water access port 130, to migrate across the initial dose semipermeable membrane 134 to reach the osmotic engine 108. As the water reaches the osmotic engine 108, the engine 108 swells in volume and increases the osmotic pressure differential across the initial dose semipermeable membrane 134 and pushes the piston 160 within the tube-shaped compartment defined by the tube 109 toward the distal end 186 thereof, as the expansion of the osmotic engine 108 is constrained to within the tube-shaped compartment 110. In so doing, the piston 160 displaces a volume of pharmaceutical agent within the tube-shaped compartment 110, which displaced volume of pharmaceutical agent is delivered out of the distal end 186 of the tube 109. The pharmaceutical agent is delivered at a selected initial infusion rate that is related to the thickness, composition and surface area of the initial dose semipermeable membrane 134. In the case wherein the initial dose semipermeable membrane 134 is implanted in a fully hydrated state, the pharmaceutical agent within the tube-shaped compartment is quickly delivered to the patient at the selected initial infusion rate. If the initial dose semipermeable membrane 134 is not pre-hydrated, the delivery of the pharmaceutical agent may be delayed until the membrane 134 becomes at least partially hydrated from water from the patient implant site. Until at least the first impermeable membrane cans 114 is breached, the only water that reaches the osmotic engine 108 enters the pump 100 through the initial water access port 130 to cross the initial dose semipermeable membrane 134.

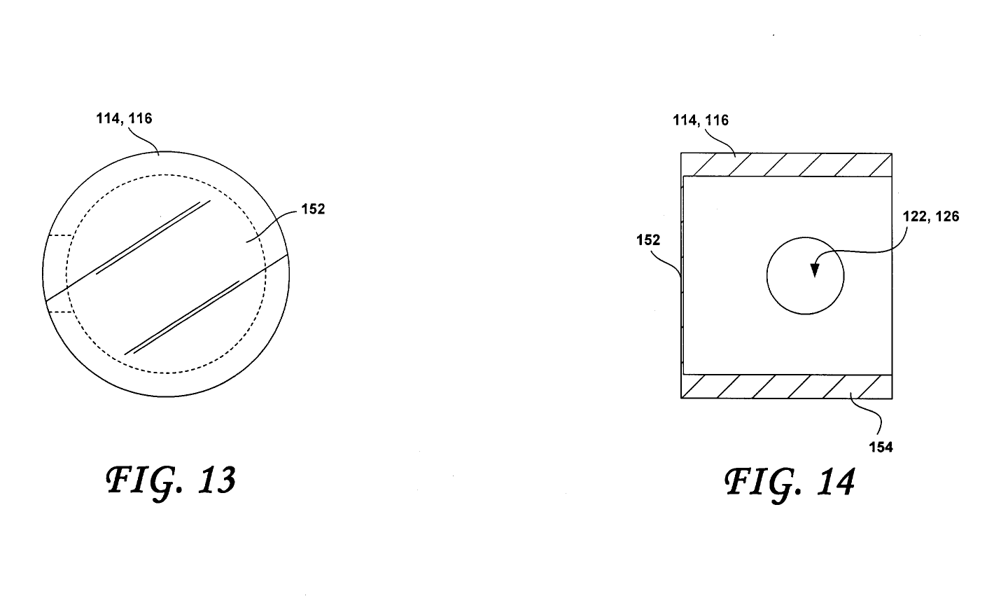

As shown in FIG. 4, the membrane assembly 112 includes a first semipermeable membrane 120 and a second semipermeable membrane 124. The diameter of the semipermeable membranes 120, 124 is directly proportional to the flow rate of the pump of an embodiment of the present invention. As shown, the first semipermeable membrane 120 may be (but need not be) vertically offset from the second semipermeable membrane 124 in the membrane enclosure 112. Reference is now made to FIGS. 13 and 14, of which FIG. 13 is a plan view of an impermeable membrane can 114, 116 and of which FIG. 14 is a side view of the impermeable membrane can 114, 116 of FIG. 13. As shown therein, the cans 114, 116 include a cylindrical sidewall 154 and a through bore defined therein. Specifically, the sidewall of the first impermeable membrane can 114 defines a first through bore 122 and the sidewall of the second impermeable membrane can 116 defines a second through bore 126. An impermeable membrane 152 (shown in FIGS. 13 and 14 in its intact state) spans one of the free ends of each of the cans 114, 116. The impermeable membranes 152, according to an embodiment of the present invention, are impermeable at least to water from the patient implant site and are configured to be easily breached by the physician, as is detailed below. The impermeable membranes 152 may include or be formed of most any water impermeable material that is biologically inert, such as titanium and/or stainless steel, coated platinum or platinum-iridium for radiopacity, for example. The impermeable membranes 152 of the first and second cans 114, 116 may be surface ground to a thickness of about 1 or 2 thousandths of an inch, for example. The impermeable membranes 152 may alternatively include polyethylene, PET, PETG or PETE, for example. Preferably, the impermeable membranes 152 are radiopaque, so as to be visible under fluoroscopy, once the pump 100 is implanted. For example, a layer of radiopaque material may be sputtered or otherwise deposited on the impermeable membranes 152, to render them visible under fluoroscopy. Preferably, the impermeable membranes 110 are adapted to be breached by the physician or clinician, using a dose escalation pen (or a lancet or stylet as shown in FIGS., 26-31), or some other functionally similar device. The impermeable membranes 152 of the first and second impermeable membrane cans 114, 116 initially seal the first and second semipermeable membranes 120, 124 to prevent any water originating from the patient's implant site from crossing the semipermeable membranes 120, 124 until the impermeable membrane(s) 152 is breached, as shown at 176 in FIGS. 28-31.

Returning now to FIGS. 3-5, the first and second impermeable membrane cans 114, 116 are stacked within the membrane enclosure 112 such that the respective through bores 122, 126 thereof are aligned with the first and second semipermeable membranes 120, 122, respectively. Specifically, the first through bore 122 defined in the first impermeable membrane can 114 is aligned with the first semipermeable membrane 120 and the second through bore 126 defined in the second impermeable membrane can 116 is aligned with the second semipermeable membrane 124. Moreover, the impermeable membrane 152 of the first impermeable membrane can 114 is disposed adjacent the primary water access port 132, whereas the second impermeable can 116 is disposed under the first impermeable membrane can 114 and oriented such that the impermeable membrane thereof is immediately adjacent the first impermeable membrane can 114. Although the present figures show the pump 100 of an embodiment of the present invention equipped with two impermeable membrane cans 114, 116, the claimed inventions are not limited thereto, as a single or a greater number of impermeable membrane cans may be used along with a corresponding number of semipermeable membranes.

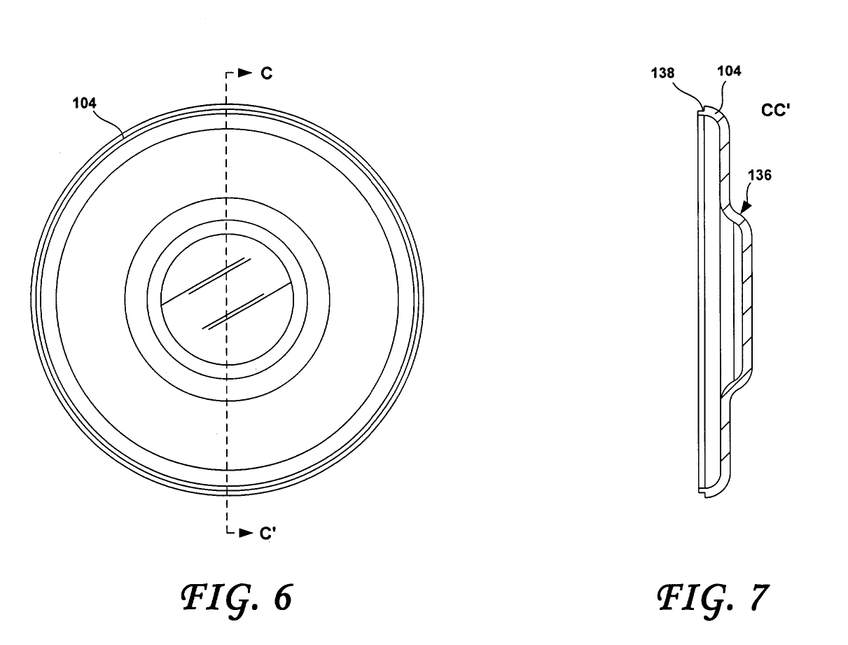

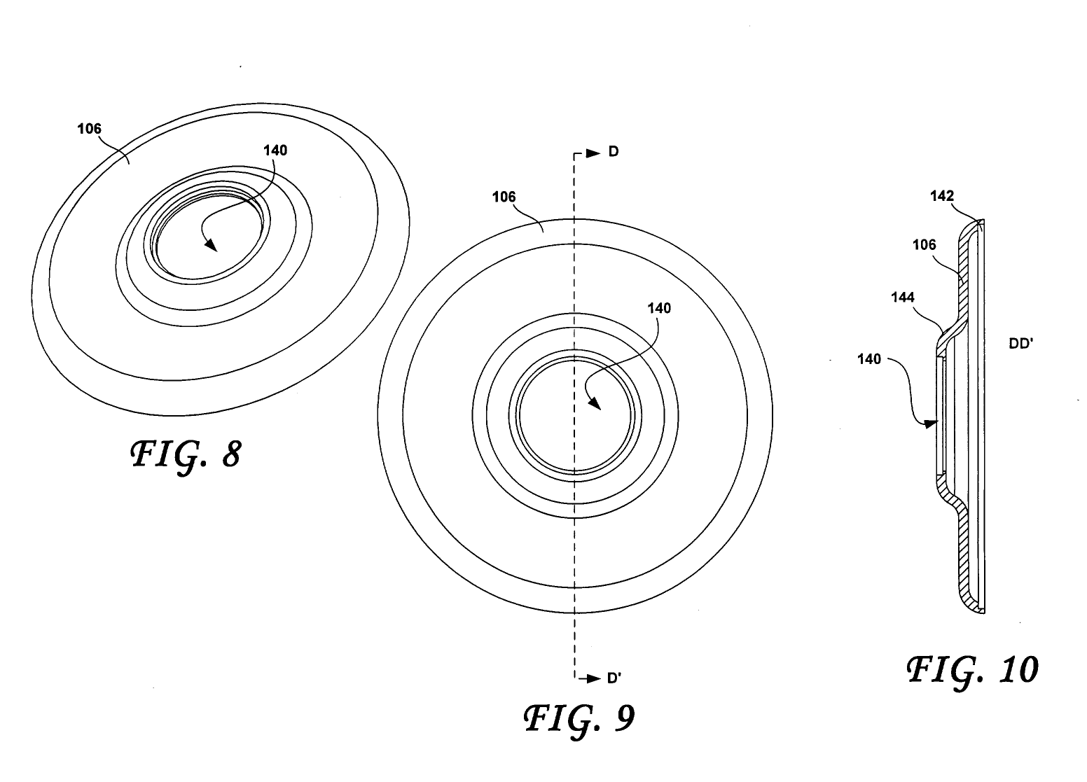

FIG. 6 is a plan view of the second half 104 of the osmotic pump housing 101, according to an embodiment of the present invention and FIG. 7 is a cross sectional view thereof, taken along lines CC′. As shown therein, the second half 104 of the pump housing 101 may have a generally saucer-like shape. Indeed, the second half 104 of the housing 101 may have a generally circular outline and may define a bulge 136 therein to accommodate a portion of the osmotic engine 108 therein. The rim of the second half 104 (See FIG. 10) of the pump housing 101 also defines an indentation 138 adapted to mate with a corresponding feature defined by the rim of the first half 106 of the pump housing 101. FIG. 8 is a perspective view of the first half 106 of the osmotic pump housing 101 according to an embodiment of the present invention, whereas FIG. 9 is a plan view and FIG. 10 is a cross-sectional view thereof, taken along lines DD′. As shown in the perspective view of FIG. 10, an opening 140 is defined in the also generally saucer-shaped first half 106 of the osmotic pump housing 101. The opening 140 may be centered in the housing half 106 and concentric with the generally circular outline thereof, as shown in FIG. 9. The opening 140 is preferably dimensioned so as to closely fit the membrane enclosure 112. As shown in FIG. 10, the first half 106 of the pump housing 101 may define a bulge 144 that increases the interior volume of the pump 100 when the first and second housing halves 106, 104 are mated to one another.

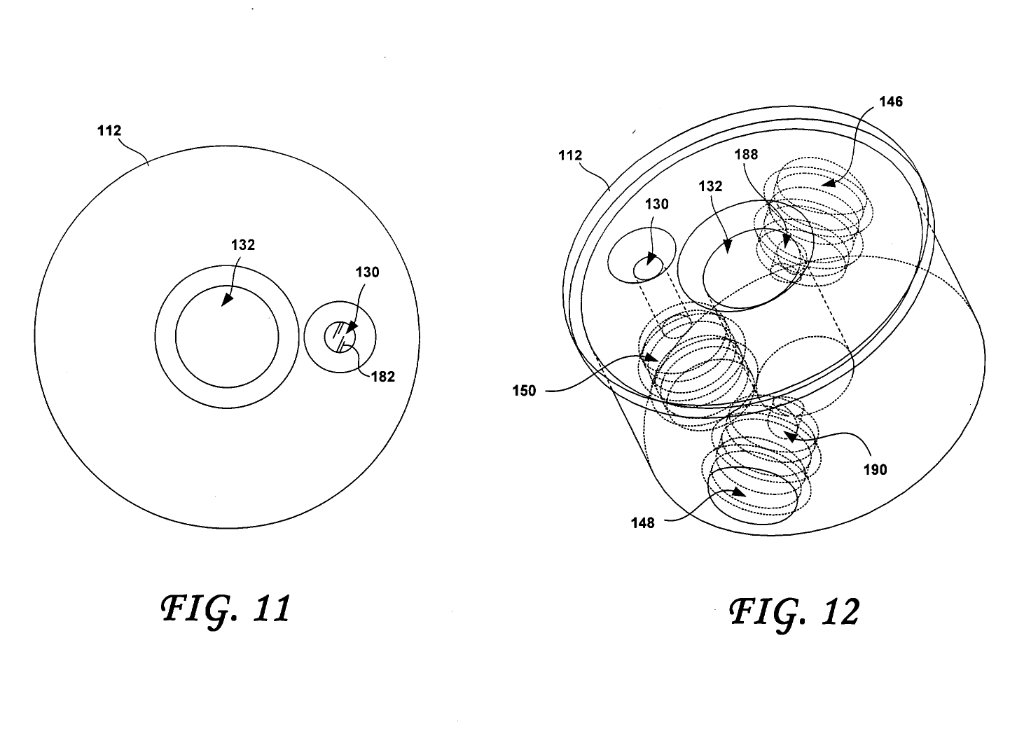

FIG. 11 is a plan view of an embodiment of the membrane housing 112, according to an embodiment thereof, whereas FIG. 12 is a perspective view of the membrane housing of FIG. 11, showing the semipermeable membrane wells in dashed lines. Considering now FIGS. 11 and 12 collectively, the membrane enclosure 112 may be shaped as a cylinder dimensioned to fit within the osmotic engine 108 and the opening 140 in the first housing half 106. The primary water access port 132 may be a bore partially through the membrane enclosure 112. However, to best control the flow of water form the patient implant site to the osmotic engine 108, the bore defined within the membrane enclosure 112 should not run the entire length of the membrane enclosure 112. Indeed, the only water paths from the implant site to the osmotic engine should be through the initial dose semipermeable membrane well 150, through the first semipermeable membrane well 146 and/or through the second semipermeable membrane well 150. In contrast, the combination of the initial water access port 130 and the initial dose semipermeable well 150 runs the entire length of the membrane enclosure 112, as also shown in FIG. 5. Indeed, once the pump 100 is implanted in the patient and any impermeable membrane that may span the initial water access port 130 is breached, a water path to the osmotic engine 108 may be defined straight through the membrane enclosure 112, as the water from the implant site migrates across the initial dose semipermeable membrane (shown at 134 in FIG. 5) fitted within the initial dose semipermeable membrane well 150.

First and second semipermeable membranes 120, 124 (shown in FIG. 4) are fitted within the first and second semipermeable membrane wells 146, 148, respectively. According to an embodiment of the present invention, when the impermeable membrane 152 of the first impermeable membrane can 114 is breached (as shown at 176 in FIGS. 28, 29 and 31), water from the implant site may enter the primary access port 132 and travel through the first through bore 122 of the first impermeable membrane can 114. From there, the water may travel through a first passageway 188, defined between primary water access port 132 and first semipermeable membrane well 146. After crossing the first semipermeable membrane 120 disposed in the well 146, the water reaches the osmotic engine 108. This first water path is shown at 178 in FIGS. 28, 29 and 31. As the water reaches the osmotic engine 108, the engine 108 swells in volume due to the osmotic pressure differential across the first semipermeable membrane 120 and pushes the piston 160, 162 within the tube-shaped compartment 110 defined within the tube 109 toward the distal end 186 thereof. In so doing, the piston 160, 162 displaces a volume of pharmaceutical agent within the tube-shaped compartment 110, which displaced volume of pharmaceutical agent is delivered out of the distal end 186 of the tube 109. The pharmaceutical agent is delivered at a selected first infusion rate that is related to the thickness, composition and surface area of the first semipermeable membrane 120 and that of the initial dose semipermeable membrane 134.

Similarly, when the impermeable membrane 152 of the second impermeable membrane can 116 is breached (as shown at 177 in FIGS. 28, 29 and 31), water from the implant site may enter the primary access port 132 and travel through the second through bore 126 of the second impermeable membrane can 116. From there, the water may travel through a second passageway 190, defined within the enclosure 112 between the primary water access port 132 and the second semipermeable membrane well 148. After crossing the second semipermeable membrane 124 disposed in the well 148, the water reaches the osmotic engine 108. This water path is shown at 180 in FIG. 31. As the water reaches the osmotic engine 108, the engine 108 swells in volume due to the osmotic pressure differential across the second semipermeable membrane 124 and pushes the piston 160, 162 within the tube-shaped compartment 110 defined by the tube 109 toward the distal end 186 thereof. In so doing, the piston 160 displaces a volume of pharmaceutical agent within the tube-shaped compartment 110, which displaced volume of pharmaceutical agent is delivered out of the distal end 186 of the tube 109. The pharmaceutical agent is delivered at a selected second infusion rate that is related to the thickness, composition and surface area of the second semipermeable membrane 124, the thickness, composition and surface area of the first semipermeable membrane 120 and the thickness, composition and surface area of the initial dose semipermeable membrane 134. Indeed, the infusion rate of the pump 100 is related to which of the semipermeable membranes 134, 120 and/or 124 are currently exposed to the patient. If only the initial dose semipermeable membrane 134 is exposed to the patient, the infusion rate may be related only to the characteristics of the initial dose semipermeable membrane 134. If both the initial dose semipermeable membrane 134 and the first semipermeable membrane 120 are exposed to the patient, the pump infusion rate may be related to the characteristics of both the initial dose and first semipermeable membranes 134, 120. In other words, the total infusion rate of the pump 100 of an embodiment of the present invention in the state wherein both the initial dose semipermeable membrane 134 and the first semipermeable membrane 120 are breached, may be approximated as the sum of the individual infusion rates contributed by each of the semipermeable membranes 134 and 120. If the initial dose semipermeable membrane 134, the first semipermeable membrane 120 and the second semipermeable membrane 124 are exposed to the patient, the pump infusion rate may be related to the characteristics of the initial dose, the first and the second semipermeable membranes 134, 120 and 124. In other words, the total infusion rate of the pump of an embodiment of the present invention in the state wherein the impermeable membranes 134, 120 and 124 are breached, may be approximated as the sum of the individual infusion rates contributed by each of the semipermeable membranes 134, 120 and 124.

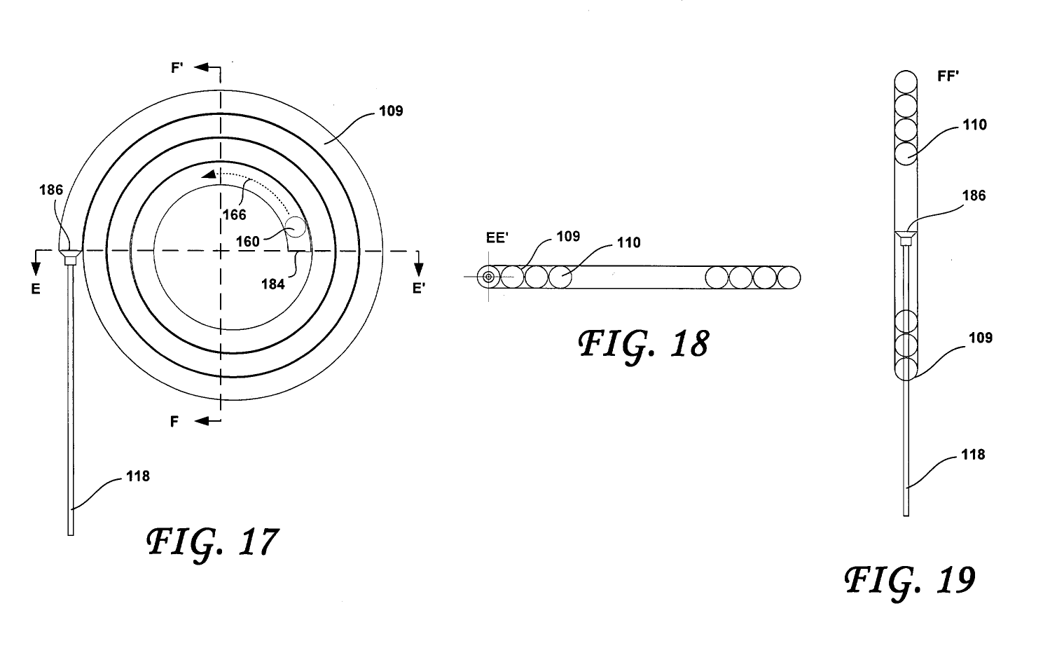

FIG. 17 is a plan view of the coiled tube 109, according to an embodiment of the present invention, FIG. 18 is a cross-sectional view of the tube 109 of FIG. 17, taken along line EE′ and FIG. 19 is a cross-sectional view thereof, taken along line FF′. According to an embodiment of the present invention, the piston 160 may initially (upon implantation) be disposed within the tube-shaped compartment 110 near the proximal end 184 of the tube 109. As the osmotic engine expands in volume, the only available volume for such expansion is within the tube-shaped compartment 110. Therefore, the expansion of the osmotic engine 108 forces the piston 160 to travel through the coiled tube 109 in the direction of arrow 166, which causes a volume of pharmaceutical agent to be delivered to the patient out of the distal end 186 of the tube 109. A catheter ID (inner diameter) tube 118 may be fitted onto the distal end 186 of the tube 109, which facilitates coupling the catheter 102 thereto. As shown, the tube 109 may be coiled a number of times around the membrane enclosure 112. In the embodiment shown in FIGS. 17-19, the tube 109 is coiled four times around the membrane enclosure 112 (not shown in FIGS. 17-19), although a lesser or greater number of coils may readily be implemented.

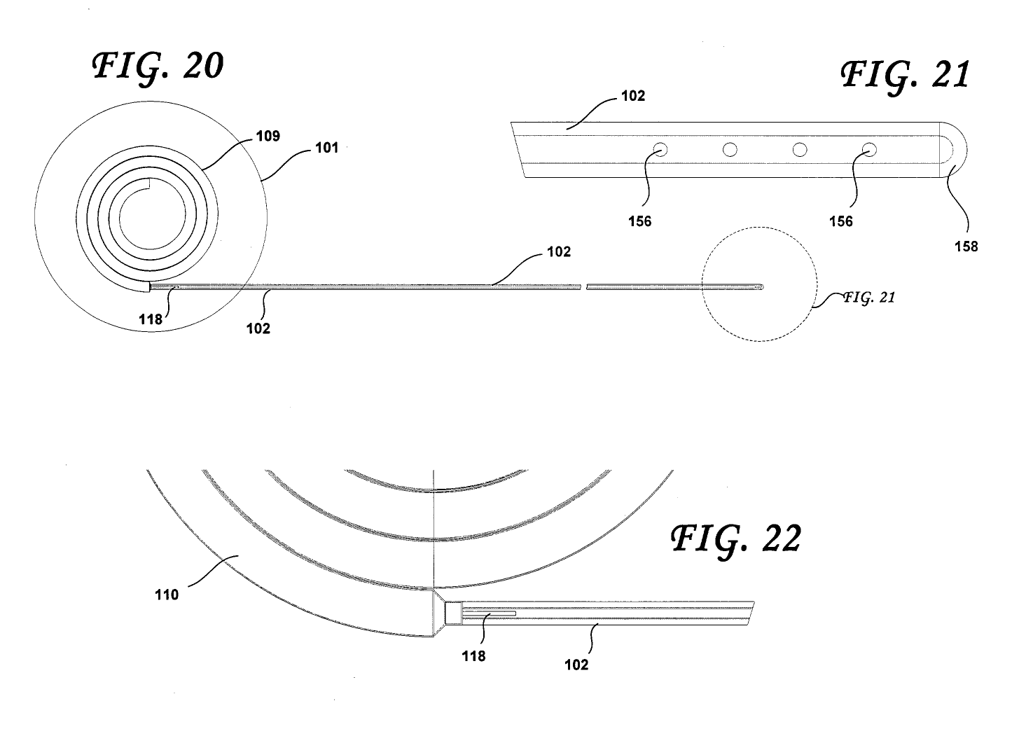

FIG. 20 illustrates the tube 109 coupled to a catheter 102, according to an embodiment of the present invention. FIG. 21 illustrates the distal tip of the catheter of FIG. 20, according to an embodiment of the present invention and FIG. 22 illustrates the manner in which the catheter may couple to the catheter ID tube 118. In FIG. 20, the outline of the pump housing 101 is shown for reference purposes. The catheter 102 is used to deliver the pharmaceutical agent from the catheter ID tube 118 to the target area within the patient's body. The catheter 102 may be visible under fluoroscopy over its length, thereby enabling the physician to trim the catheter to the desired length. Alternatively, the catheter 102 may include distal radiopaque markers, for example. As shown in FIG. 21, the distal tip 158 of the catheter 102 may included a rounded, atraumatic tip. A plurality of pharmaceutical agent openings 158 may be defined through the catheter wall, from the internal lumen thereof to the patient. As shown in FIG. 22, the catheter ID may be fitted over the catheter ID tube 118 using a friction fit and/or suitable biocompatible adhesive(s), for example. Any suitable radio opaque material may be used to render all or a portion or selected portions of the catheter 102 radio opaque. For example, the catheter 102 may be formed of silicone or polyurethane and may be doped with barium sulfate, for example. The length of the catheter 102 may be most any therapeutically effective length. A longer length, however, increases the dead space therein and delays the effusion of the pharmaceutical agent into the patient, as it will take longer for the agent to travel the length thereof. For example, the catheter 102 may be about 5cm to about 100 cm in length. More preferably, the catheter 102 may be about 10 cm to about 30 cm in length. More preferably still, the catheter 012 may be about 15 cm to about 25 cm in length. For example, the catheter 102 may be about 20 cm in length. The internal diameter (ID) of the infusion lumen of the catheter 102 may be selected within the range of about 0.001 inches to about 0.010 inches. The walls of the catheter 102 may be about 0.001 inches to about 0.006 inches in thickness. According to an embodiment of the present invention, the outer diameter (OD) of the catheter 102 may be selected between about 0.024 inches and about 0.066 inches in thickness, for example.

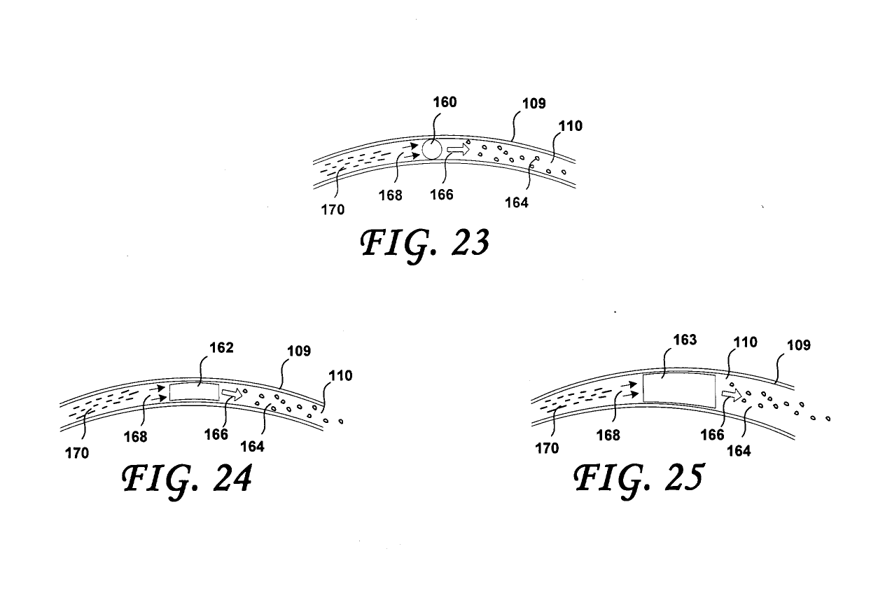

FIGS. 23-25 are cross sections of the tube 109, showing various designs for the piston within the tube shaped compartment 110. Considering now FIGS. 23-25 collectively, the piston of the osmotic pump 100 of an embodiment of the present invention may be spherical, as shown at 160, cylindrical as shown at 162 or may approximate a conical section as shown at 163, although other shapes are possible. A spherical shape minimizes the contact points of the piston 160 with the tube-shaped compartment 110, thereby enabling the piston 160 to travel through the compartment 110, even as the radius of curvature thereof changes form the proximal end 184 to the distal end of the tube 109. Reference 170 represents slurry from the osmotic engine 108. Indeed, reference 170 may be considered to be an extension of the osmotic engine 108, as it swells with water from the patient implant site through the semipermeable membranes 134, 120 and/or 124. As the osmotic engine 108 swells in volume, it exerts a force 168 on the piston 160, 162 or 163, forcing it to travel within the tube-shaped compartment 110 in the direction of arrow 166. In so doing, the piston 160, 162, 163 displaces a corresponding volume of pharmaceutical agent 164. The piston 160, 162, 163 may include stainless steel, nylon or an elastomer, for example. When the piston has a cylindrical shape, as shown on FIG. 24 at 162, the piston 162 may be formed of an elastomeric substance, such as butyl rubber, for example. Such a cylindrical piston 162 may then deform to match the radius of curvature of the tube-shaped compartment 110. The inner diameter of the tube 109 (that is, the diameter of the tube-shaped compartment 110) may be constant over the length of the tube 109 or may become larger or smaller over its length. In the latter case, the piston 163 may assume a truncated conical shape, in which a proximal end thereof is smaller than a distal end thereof (or vice-versa), to match the change in inner diameter of the tube-shaped compartment 110. To prevent the tube 109 from compressing, binding and/or kinking as the osmotic engine 108 swells, the coiled tube 109 may be encased in a hard substance, such as epoxy, for example.

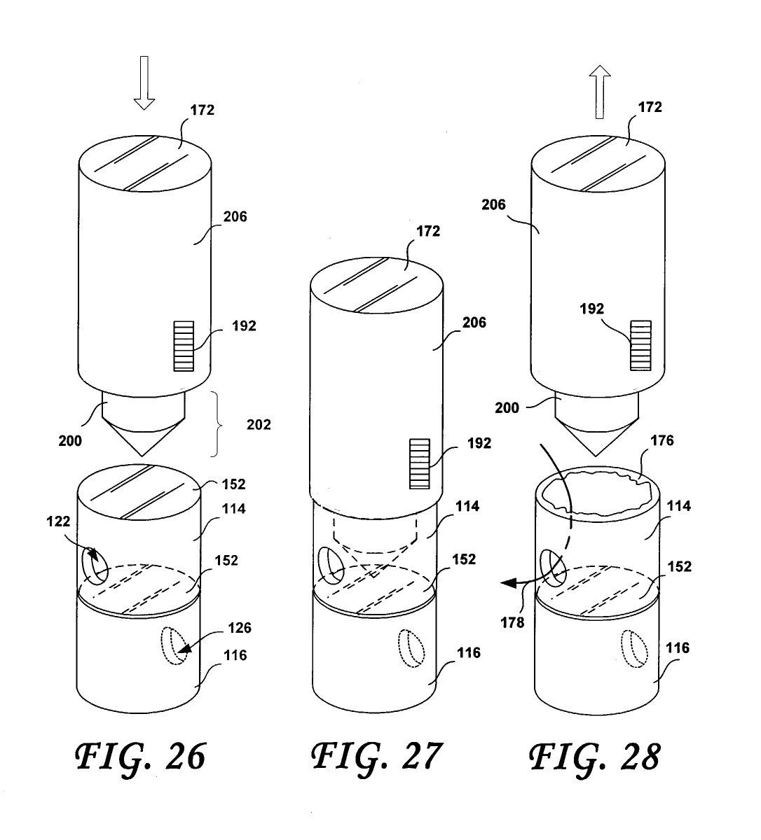

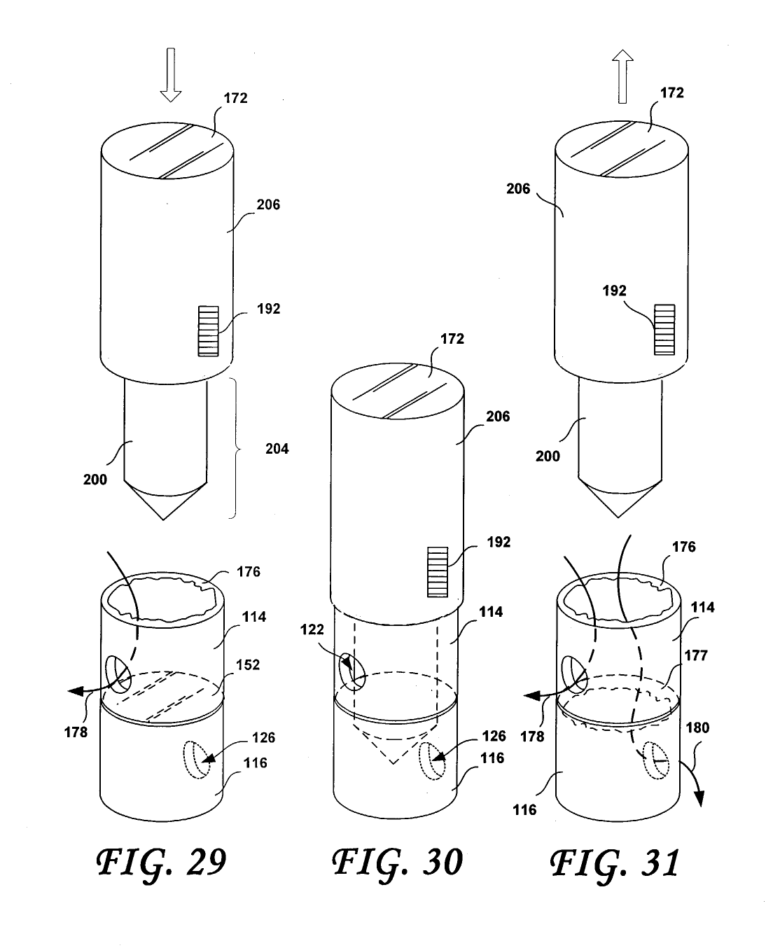

FIGS. 26-28 shows steps of a method by which the impermeable membrane 152 of the first impermeable membrane can 114 may be breached so as to escalate a dose of pharmaceutical agent delivered to the patient, according to an embodiment of the present invention. FIGS. 29-31 shows further steps of the method by which the impermeable membrane 152 of the second impermeable membrane can 116 may be breached so as to further escalate the dose of pharmaceutical agent delivered to the patient, according to an embodiment of the present invention. While any device may be used to breach the impermeable membranes 152, a dose escalation pen or stylet 172 similar to that shown in FIGS. 26-31 may be advantageously used. An actuator 192, such as a thumb actuated wheel, may be coupled to a pointed extendible portion 200 of the pen 172. Actuating the actuator 192 may cause the pointed and extendible portion 200 to extend in length from a first length 202 shown in FIGS. 26-28, to a second length 204 shown in FIGS. 29-31. At some time after implantation of the pump 100, the patient may require a greater dose of pharmaceutical agent than provided by the initial dose, which initial dose is driven by the osmotic engine 108 swelling in response to water entering the initial water access port 132. Without removing the pump 100 from the patient, the physician may, according to an embodiment of the present invention, use a dose escalation pen or stylet to increase the effusion rate of the pharmaceutical agent from the pump 100 in a simple office or outpatient procedure.

For clarity of illustration, only the first and second impermeable membrane cans 114, 116 of the pump 100 are shown in FIGS. 26-31. In the state illustrated in FIG. 26, the impermeable membranes 152 prevent any water from the patient implant site from reaching the first and second semipermeable membranes 120, 124. When the physician wishes to increase the dose of pharmaceutical agent delivered to the patient, he or she may use the dose escalation pen 172 in a configuration wherein the pointed extendible portion 200 thereof is extended only to the first length 202. By inserting the portion 200 through the patient's skin under fluoroscopic, ultrasonic or manual (palpation) guidance, for example, the physician may breach the impermeable membrane 152 of the first impermeable membrane can 114, as shown at FIG. 27. Preferably, the first length 202 of the extendible portion 200 is selected so as to breach only the impermeable membrane 152 of the first can 114, and not that of the second can 116. Preferably, the outer diameter of the extendible portion 200 is slightly smaller than the outer diameter of the cans 114, 116, to enable the dose escalation pen 172 to create a wide opening when breaching the impermeable membranes 152. Similarly, the handle portion 206 of the pen 172 should have a diameter that is slightly larger than the outer diameter of the cans 114, 116, to limit the travel of the extendible portion 200 within the cans 114, 116. As shown in FIG. 28, once the dose escalation pen 172 is retracted after the impermeable membrane of the first can 114 is breached, a first water path 178 is created, from the patient implant site through the first impermeable membrane can 114, through the first through bore 122 thereof, across the first semipermeable membrane 120 to the osmotic engine 108. In this state of the pump 100, water may now reach the osmotic engine 108 through the initial water access port 132 and through the first impermeable membrane can 114.

Turning now to FIGS. 29-31, when the patient requires an even greater dose of pharmaceutical agent, the physician may actuate the actuator 192 to change the length of the extendible portion 200 to the second length 204, which second length 204 is sufficient to penetrate the first can 114 and breach the impermeable membrane 152 of the second impermeable membrane can 116, as shown at 177 FIG. 31. After the dose escalation pen 172 is retracted as shown at FIG. 31, a second water path 180 is created. The second water path 180 runs from the patient implant site through the first impermeable membrane can 114, through the breached impermeable membrane 152 of the second can 116, through the second through bore 126 of the second can 116, across the second semipermeable membrane 124 to the osmotic engine 108. In this state of the pump 100, water may now reach the osmotic engine 108 through the initial water access port 132, through the first impermeable membrane can 114 as well as through the second impermeable membrane can 116.

The tube-shaped compartment 110 of the pump 100 may be pre-loaded with one or more pharmaceutical agents. 30. For example, the pharmaceutical agent may be therapeutically effective for one or more of the following therapies: pain therapy, hormone therapy, gene therapy, angiogenic therapy, anti-tumor therapy, chemotherapy, allergy therapy, hypertension therapy, antibiotic therapy, bronchodilation therapy, asthmatic therapy, arrhythmia therapy, nootropic therapy, cytostatic and metastasis inhibition therapy, migraine therapy, gastrointestinal therapy and/or other pharmaceutical therapies.

For example, the pharmaceutical agent may include an opioid, a morphine-like agonist, a partial agonist, an agonist-antagonist and/or an alpha 2-adrenoreceptor agonist. Advantageously, the pharmaceutical agent may include morphine, hydromorphone, levorphanol, methadone, fentanyl, sufentanil, buprenorphine, pentazocine and/or butorphanol, for example. The pharmaceutical agent may, for example, include an analgesic agent such as Dihydrocodeine, Hydromorphone, Morphine, Diamorphine, Levorphanol, Butorphanol, Alfentanil, Pentazocine, Buprenorphine, Nefopam, Dextropropoxyphene, Flupirtine, Tramadol, Oxycodone, Metamizol, Propyphenazone, Phenazone, Nifenazone, Paracetamol, Phenylbutazone, Oxyphenbutazone, Mofebutazone, Acetyl Salicylic Acid, Diflunisal, Flurbiprofen, Ibuprofen, Diclofenac, Ketoprofen, Indomethacin, Naproxen, Meptazinol, Methadone, Pethidine, Hydrocodone, Meloxicam, Fenbufen, Mefenamic Acid, Piroxicam, Tenoxicam, Azapropazone, Codein, Bupivacaine, Ketamine, Meperidine and/or [D-Ala2,D-Leu5]enkephalin (DADL). The pharmaceutical agent may also include analgesic that is an alpha-2 adrenergetic agonist such as Clonidine, Tizadine, ST-91, Medetomidine, Dexmedetomidine and/or related alpha-2 adrenergetic agonists. The analgesic may also include an N-methyl-D-aspartate (NMDA) receptor agonist including Dexmethorphan, Ifenprodil, (+)-5-methyl-10,11-dihydro-5H-dibenzo[a,d]-cyclohepten-5,10-imine (MK-801), and/or related NMDA agonists. The analgesic may also include a somatostatin analog selected including Octreotide, Sandostatin, Vapreotide, Lanreotide, and/or related Somatostatin analogs, for example. Alternatively, the pharmaceutical agent may include a non-opioid analgesic such as Ketorolac, super oxide dismutase, baclofen, calcitonin, serotonin, vasoactive intestinal polypeptide, bombesin, omega-conopeptides, and/or related non-opioid analgesics, for example. The pharmaceutical agent in the compartment 310 may be dissolved in an aqueous solution.

For pain therapy, a preferred pharmaceutical agent is Sufentanil. In that case wherein the pharmaceutical agent is (or includes) Sufentanil that is dissolved in an aqueous medium, it has been found that the solubility of the Sufentanil within the aqueous solution increases with increasing acidity of the medium. For example, the pumps according to embodiments of the present invention may be configured to deliver Sufentanil at up to about 1500 μg/day, at a concentration of up to about 500,000 μg/ml, when the Sufentanil is dissolved in an acidic aqueous medium.

### EXAMPLE

A pump according to an embodiment of the present invention may include a pharmaceutical agent compartment 310 having a volume of 500 μl (microliters). A compartment 310 of this volume may contain 500 μl of pharmaceutical agent solution, the solution including 250,000 μg of Sufentanil dissolved in an acidic aqueous medium. Therefore, about 1500 μg/day of such pharmaceutical agent solution may be delivered to the patient over a treatment period spanning about 167 days. Implanted into a patient, such a pump would deliver about 3 μl of pharmaceutical agent solution to the patient per day, each such 3 μl of pharmaceutical agent solution containing about 1500 μl of Sufentanil.

The pharmaceutical agent may also include an anti-allergic agent including Pheniramine, Dimethindene, Terfenadine, Astemizole, Tritoqualine, Loratadine, Doxylamine, Mequitazine, Dexchlorpheniramine, Triprolidine and/or Oxatomide, for example. The pharmaceutical agent may include one or more anti-hypertensive agents, such as Clonidine, Moxonidine, Methyldopa, Doxazosin, Prazosin, Urapidil, Terazosin, Minoxidil, Dihydralalzin, Deserpidine, Acebutalol, Alprenolol, Atenolol, Metoprolol, Bupranolol, Penbutolol, Propranolol, Esmolol, Bisoprolol, Ciliprolol, Sotalol, Metipranolol, Nadolol, Oxprenolol, Nifedipine, Nicardipine, Verapamil, Diltiazim, Felodipine, Nimodipine, Flunarizine, Quinapril, Lisinopril, Captopril, Ramipril, Fosinoprol and/or Enalapril, for example. Alternatively, the pharmaceutical agent may include an antibiotic agent such as Democlocycline, Doxycycline, Lymecycline, Minocycline, Oxytetracycline, Tetracycline, Sulfametopyrazine, Ofloaxcin, Ciproflaxacin, Acrosoxacin, Amoxycillin, Ampicillin, Becampicillin, Piperacillin, Pivampicillin, Cloxacillin, Penicillin V, Flucloxacillin, Erythromycin, Metronidazole, Clindamycin, Trimethoprim, Neomycin, Cefaclor, Cefadroxil, Cefixime, Cefpodoxime, Cefuroxine, Cephalexin and/or Cefradine, for example. Bronchodialotors and anti-asthmatic agents may also be pre-loaded into the tube-shaped compartment 110, including Pirbuterol, Orciprenaline, Terbutaline, Fenoterol, Clenbuterol, Salbutamol, Procaterol, Theophylline, Cholintheophyllinate, Theophylline-ethylenediamine and/or Ketofen, for example. Anti-arrhythmic agents may also be pre-loaded into the pump 100, including Viquidil, Procainamide, Mexiletine, Tocainide, Propafenone and/or Ipratropium, for example. The pharmaceutical agent may alternatively include a centrally acting substance such as Amantadine, Levodopa, Biperiden, Benzotropine, Bromocriptine, Procyclidine, Moclobemide, Tranylcypromine, Tranylpromide, Clomipramine, Maprotiline, Doxepin, Opipramol, Amitriptyline, Desipramine, Imipramine, Fluroxamin, Fluoxetin, Paroxetine, Trazodone, Viloxazine, Fluphenazine, Perphenazine, Promethazine, Thioridazine, Triflupromazine, Prothipendyl, thiothixene, Chlorprothixene, Haloperidol, Pipamperone, Pimozide, Sulpiride, Fenethylline, Methylphenildate, Trifluoperazine, Oxazepam, Lorazepam, Bromoazepam, Alprazolam, Diazepam, Clobazam, Buspirone and/or Piracetam, for example. Cytostatics and metastasis inhibitors may also be pre-loaded within the pump 100 of an embodiment of the present invention, including Melfalan, Cyclophosphamide, Trofosfamide, Chlorambucil, Busulfan, Prednimustine, Fluororacil, Methotrexate, Mercaptopurine, Thioguanin, Hydroxycarbamide, Altretamine and/or Procarbazine, for example. Other pharmaceutical agents that may be pre-loaded include anti-migrane agents such as Lisuride, Methysergide, Dihydroergotamine, Ergotamine and/or Pizotifen or gastrointestinal agents such as Cimetidine, Famotidine, Ranitidine, Roxatidine, Pirenzipine, Omeprazole, Misoprostol, Proglumide, Cisapride, Bromopride and/or Metoclopramide.

Embodiments of the present invention also include kits, including an implantable osmotic pump 100, a catheter 102 configured to attach to the pump 100 and/or dose escalation pen(s) 172 configured to breach the impermeable membranes 152 of the first and/or second cans 114, 116.

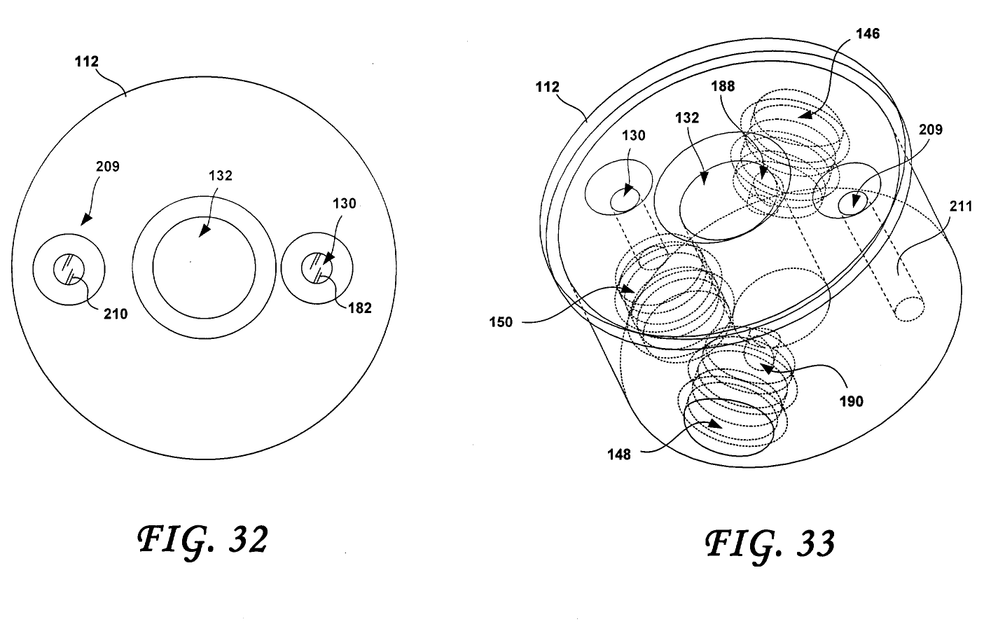

There may be instances wherein it is desired to shut the pump down. For example, an adverse reaction to the pharmaceutical agent may have occurred. FIGS. 32 and 33 are plan and perspective views, respectively, of a membrane enclosure 112, according to embodiment of the present invention that addresses this need. As shown therein, the membrane enclosure 112 of FIGS. 32 and 33 is identical to the membrane enclosure of FIGS. 11 and 12, but for the presence of the structure referenced at 209. Reference 209 denotes an OFF switch that is configured to enable the physician to nullify or substantially nullify the osmotic pressure differential across any and all semipermeable membranes such as shown at 120 or 124. The OFF switch 209 includes an OFF switch impermeable membrane 210 and an OFF switch impermeable lumen 211. When and if the OFF switch impermeable membrane 210 is breached, fluid from the patient's implant site flows into the OFF switch lumen 211, bypasses the semipermeable membranes, and flows directly to the osmotic engine 108. Thus, any existing osmotic pressure that may have developed across such semipermeable membranes is reduced to zero or substantially zero, which correspondingly reduces the pump's driving force and reduces the delivery rate of the pharmaceutical agent to zero or about zero. The pump may then be explanted from the patient at will or may simply be left in place.

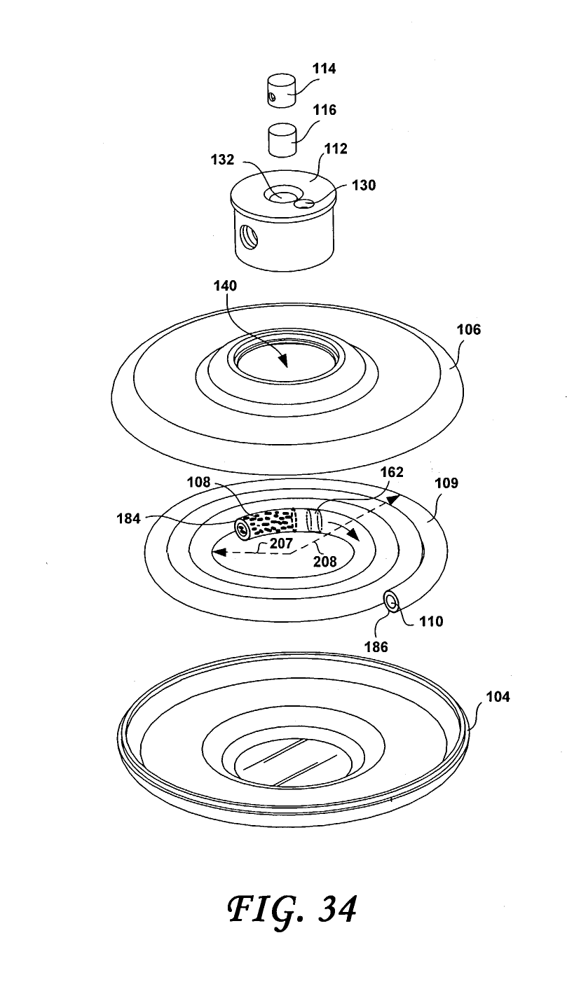

FIG. 34 is an exploded view of another embodiment of an osmotic pump according to an embodiment of the present invention. FIG. 34 is similar to FIG. 1, but for the osmotic engine 108. Accordingly, the description of the structures in FIG. 1 that are identical to structures in FIG. 34 is incorporated herein by reference. In FIG. 34, at least a portion of the osmotic engine is disposed within the tube 109, at or near the proximal end 184 thereof. The tube, in this case, is preferably rigid and may be formed of, for example, stainless steel or titanium. In this manner, the expansion of the osmotic engine 108 may be entirely constrained within the tube 109, thereby pushing the piston 162 within the tube 109 toward the proximal end 186 thereof.

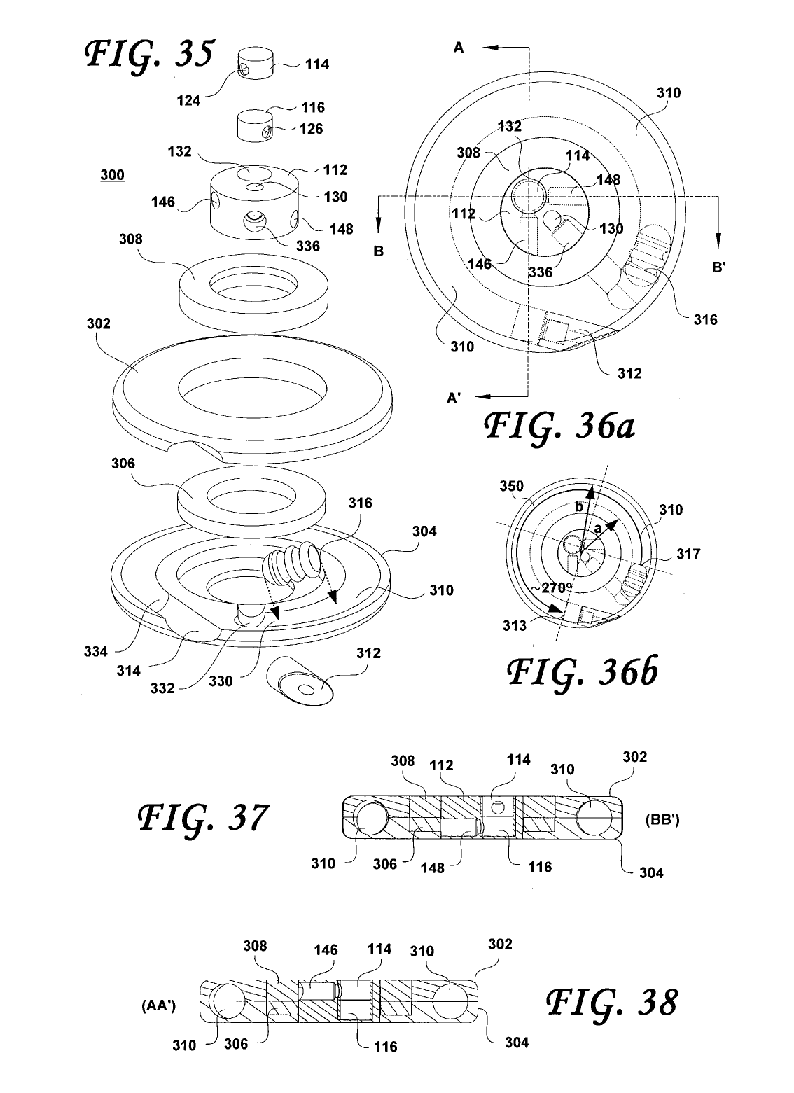

FIG. 35 is an exploded view of a three-stage osmotic pump 300, according to another embodiment of the present invention. FIG. 36 is a top view of a three stage osmotic pump according to an embodiment of the present invention, showing the internal structure thereof in dashed lines. FIGS. 37 and 38 are cross-sectional views of a three stage osmotic pump according to an embodiment of the present invention, taken along cross-sectional line BB′ and AA′ of FIG. 36. Considering now FIGS. 35-38 collectively, the constituent elements of the pump 300 that are similar to corresponding elements in FIG. 2 are identified by the same reference numerals and the detailed description thereof is omitted here. As shown, the osmotic pump 300 includes a substantially saucer-shaped housing that includes a first housing half 302 and a second housing half 304 that mates with the first housing half 302. In contradistinction to the embodiment shown in FIG. 2, the osmotic pump 300 of FIG. 35 does not include a tube, such as tube 109. Instead, when mated together, the first and second halves 302, 304 of the pump housing together define a tube-shaped and fluid-tight compartment 310 that is adapted to enclose a pharmaceutical agent. The compartment 310 is substantially toroidal in shape, in that it resembles a tube that curves around the osmotic engine 306, following the outer curvature of the pump housing throughout most of its length. The tube-shaped compartment 310 defines a first end 330 that is in fluid communication with the osmotic engine 306 through a passageway 332 and a second end 334 adjacent the compartment outlet 314 that is formed when the first and second halves 302, 304 of the housing are joined together.

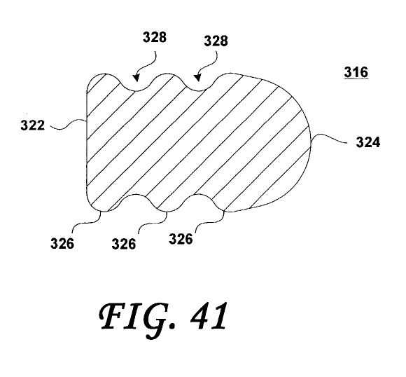

The pump 300 includes a piston 316 that is configured and adapted to travel within the compartment 310 in response to the force exerted thereon by the osmotic engine 306. As the piston 316 travels within the compartment 310, it displaces a volume of pharmaceutical agent. The piston 316, when the pump 300 is first implanted, is located adjacent the first end 330 of the compartment 310 and thereafter travels from the first end 330 toward the second end 334, displacing a volume of pharmaceutical agent as it travels. FIG. 41 shows a cross-section of an exemplary embodiment of a piston 316. As shown therein, the piston 316 may define a leading end 322 and a trailing end 324. Additionally, to reduce the surface area of the piston 316 that contacts the wall of the pharmaceutical agent compartment 310, the outer surface of the piston may define one or more throughs 328 and ridges 326, thereby further facilitating the travel of the piston 316 through the compartment 310.

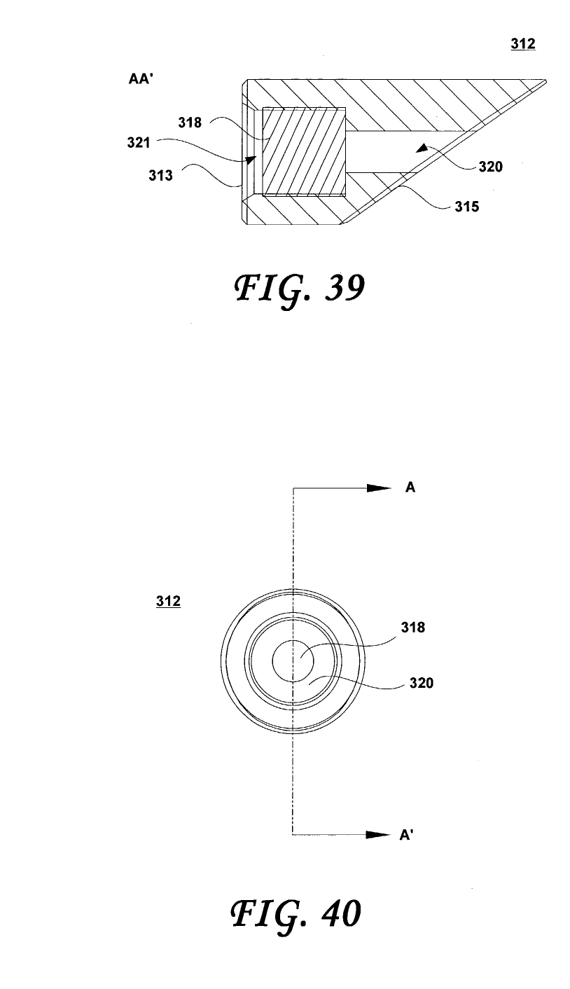

Returning now to FIG. 35, the pump 300, when configured for systemic delivery of a pharmaceutical agent (as is the case wherein the pump is implanted subcutaneously, for example), may include a filter assembly 312. The filter assembly 312 is configured to fit within the compartment outlet 314, so as to maintain the substantially circular footprint of the pump 300, as shown most clearly in FIG. 36. The structure of the filter assembly 312 is further described below, with reference to FIGS. 39 and 40. Functionally, the filter assembly 312 filters the flow of the pharmaceutical agent from the pump 300 to the implant site within the patient or to the aqueous environment in which the pump is deployed. The filter assembly 312 prevents the passage of crystallized pharmaceutical agents to the patient. Crystallized pharmaceutical agents present a danger to the patient, in that the crystallized portion may contain an excess amount of agent and may cause an overdose.

Assuming that the tube-shaped compartment 310 is substantially circular in cross-section, the volume of pharmaceutical agent that may be contained therein may be estimated by:

n/360[¼Π²(a+b)(b−a)²

where, as shown in FIG. 36b (which figure is not shown to the same scale as FIG. 36a), a is the inner radius of the compartment 310, b is the outer radius of the compartment 310 and n represents the number of degrees that the compartment 310 is coiled around the pump 300, as shown by arrow 350. As shown in the embodiment illustrated in FIG. 36b, n is about 270°, as the portion of the compartment 310 that is free to enclose pharmaceutical agent (i.e., from the leading edge 317 of the piston 316 to the proximal edge 313 of the filter assembly 312) spans about ¾ of the circumference of the pump 300.

The pump 300 may also include a ring 308. The ring 308 is preferably formed of the same material as the first and second housing halves 302, 304 such as stainless steel, titanium or alloys thereof, for example. To assemble the pump 300, the piston 316 may be placed adjacent the first end 330 of the compartment 310 and the osmotic engine 306 may be centered between the first and second housing halves 302, 304. The first and second housing halves 302, 304 may then be welded together, along the circumferential seam thereof. The first and second impermeable membrane cans 114, 116 may then be inserted into the membrane enclosure, properly aligned therein and secured thereto. The ring 308 may then be inserted into the central opening formed by the first and second housing halves 302, 304 and the semipermeable membrane enclosure 112, complete with the first and second impermeable cans 114, 116 may then be dropped into the central opening of the ring 308, taking care to align the first through bore 124 with the first semipermeable membrane well 146 and the second through bore 124 with the second semipermeable membrane well 148. The enclosure 112 may then be welded to the ring 308 and the ring 308 may be welded to the first half 302 of the pump housing (not necessarily in that order). The compartment 310 may then be filled with pharmaceutical agent (not shown in FIG. 35) and the filter assembly 312 may thereafter be fitted within the compartment outlet 314 and secured therein. Note that the initial dose semipermeable membrane fitted within the initial dose semipermeable membrane well 336 is not shown in FIGS. 35-38, nor is the first semipermeable membrane fitted within the first semipermeable membrane well 146 or the second semipermeable membrane fitted within the second semipermeable membrane well 148. The membrane enclosure 112 may also incorporate the OFF switch features shown in FIGS. 32 and 33. According to the embodiment of the present invention shown in FIGS. 35-38, the pump 300 is adapted to deliver a pharmaceutical agent or agents at three distinct rates. The first or initial rate occurs when the pump 300 is implanted within the patient and only the initial water access port 130 is in fluid communication with the fluid environment of the pump's implant site within the patient. In this configuration, water from the implant site enters the pump at 130, crosses the initial dose semipermeable membrane in the semipermeable membrane well 336 and comes into contact with the osmotic engine 306, causing the engine 306 to swell and to push the piston 316 toward the second end 334 of the compartment 310 at an initial first rate. Thereafter, the physician may puncture the impermeable membrane of the first can 114, thereby causing water form the implant site to enter therein, cross the first semipermeable membrane within the first semipermeable membrane well 146 and reach the osmotic engine 306. The delivery rate of the pump 300 is now increased from its first, initial rate to a second, larger rate, as more water from the patient implant site is reaching the osmotic engine 306, causing it to swell at a faster rate, thereby causing to piston 316 to travels within the compartment 310 at a corresponding second, faster rate. When the second impermeable membrane can 116 is breached, water from the implant site enters therein, crosses the second semipermeable membrane within the second semipermeable membrane well 148 and reaches the osmotic engine 306. The delivery rate of the pump 300 is now increased from its second rate to a third, even greater rate, as more water from the patient implant site reaches the osmotic engine 306, causing it to swell at a faster rate, thereby causing to piston 316 to travel within the compartment 310 at a third, faster rate, thus displacing a greater amount of pharmaceutical agent than either the initial or second rates.

FIG. 39 is a cross-sectional view of the filter assembly 312 of FIG. 35 and FIG. 40 is a front view of the filter assembly 312 of FIG. 35. As shown in FIGS. 35 and 39-40, the filter assembly 312 may be (but need not be) shaped as a slanted and truncated circular cylinder. The filter assembly 312 defines a proximal end 313 and a distal end 315. The assembly 312 further defines a pharmaceutical agent inlet 321 that emerges at the proximal end 313 and a pharmaceutical agent outlet 320 that emerges at the distal end of the filter assembly 312. Between the inlet 321 and the outlet 320, the filter assembly includes a filter 318. According to an embodiment of the present invention, the filter 318 may include a plug of porous material that defines a plurality of pores. The pores, according to an embodiment of the present invention, may range from about 2 microns in average pore size to about 80 microns in average pore size, for example. For example, the average pore size of the porous material of the filter 318 may be selected within the range of about 5 microns to about 20 microns.

The porous material of the filter 318 may be selected to be hydrophilic or hydrophobic, depending upon, for example, the nature of the pharmaceutical agent contained in the pump 300. The pharmaceutical agent in the compartment 310 may be dissolved in an aqueous solution. Alternatively, the pharmaceutical agent in the compartment 310 of the pump 300 may be dissolved in a non-aqueous solution, such as alcohol (benzyl alcohol, for example). In such a case, the filter assembly 318 may include a filter that is substantially hydrophobic in nature, which would allow the passage of a hydrophobic solution, but would not admit the passage of a (or a substantial amount of a hydrophilic solution such as water. Water (or substantial amounts thereof) from the patient implant site, therefore, could not get into the pump 300 and only the pharmaceutical agent could get out, into the patient. Alternatively, the porous material 318 may have hydrophilic characteristics. When the porous material 318 of the filter assembly 312 is hydrophilic, reliance is made on the pressure differential across the porous material 318 (higher on the proximal end 313 than on the distal end 315 end thereof, due to the pressure exerted by the osmotic engine 306) as well as on the pore size of the porous material 318 to limit the diffusion into the pump 300. The pore size may be selected depending upon the magnitude of the pressure differential across the filter assembly 312, the length of the filter 318, the nature of the pharmaceutical agent to be delivered (for example, some pharmaceutical agent including large-sized protein molecules such contained in many pain medications may require a filter 318 defining relatively large size pores) and the aspect ratio of the filter 318 (ratio of aggregate pore size to length of filter 318), among other factors. Suitable materials for the porous material of the filter 318 may be obtained from, for example Millipore Corp. (http://www.millipore.com), Porex Corp. (http//:www.porex.com) and others.

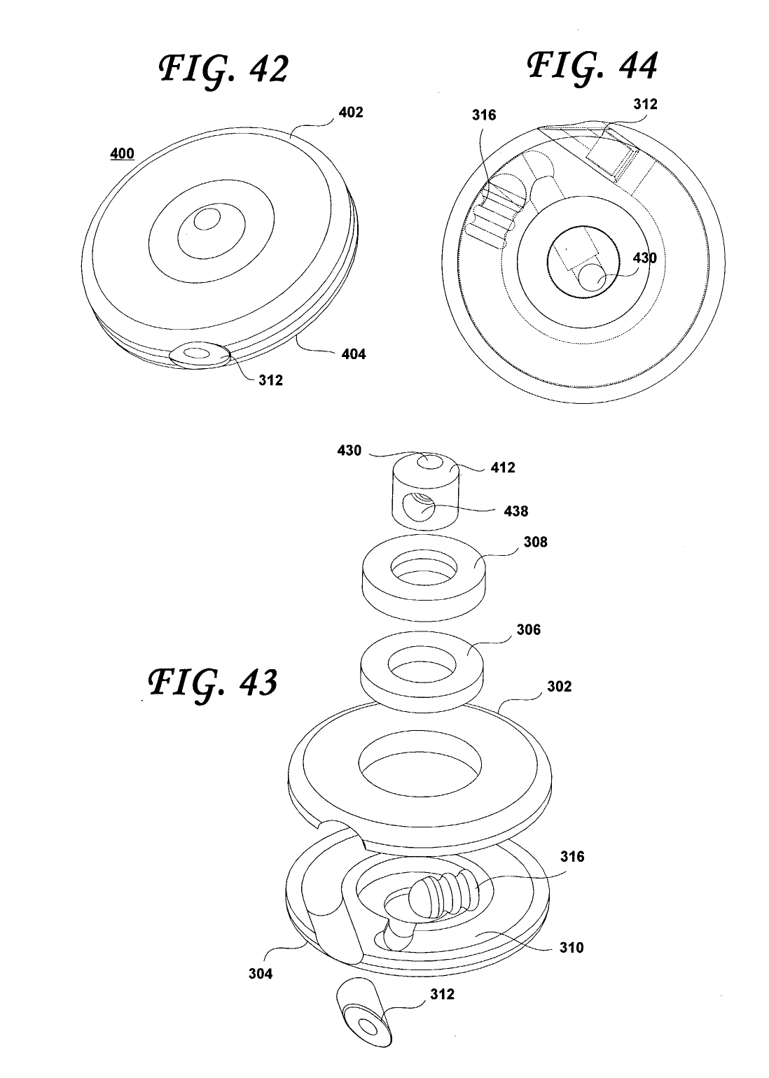

FIGS. 42, 43 and 44 show a perspective view, an exploded view and a top view of a single stage osmotic pump according to another embodiment of the present invention, with the top view of FIG. 44 showing internal components thereof in dashed lines. The pump 400 includes first and second housing halves 302, 304, filter assembly 312, piston 316, osmotic engine 306 and ring 308, each of which being similar or identical to those structures in FIGS. 35-38 referenced by the same numerals. A detailed description of these structures is, therefore, omitted here. The single-stage pump 400 may include a semipermeable membrane enclosure 412. The semipermeable membrane enclosure 412 may define a water access port 430 through which water from the patient implant site enters the pump 400. The enclosure 412 also defines a water outlet port 438, thorough which water comes into contact with the osmotic engine 306. Between the water inlet port 430 and the water outlet port 438 is disposed a semipermeable membrane. The water inlet port 430 may be covered by an impermeable membrane of stainless steel or titanium, for example. Moreover, a saturated saline solution may be present between the impermeable membrane covering the water inlet port 430 and the semipermeable membrane within the enclosure 412. Such a saturated saline solution maintains the semipermeable membrane in a hydrated state, and speeds up the initial delivery of the pharmaceutical agent contained in the compartment 310 of the pump 400 once the (optional) impermeable membrane covering the water inlet port 430 is breached. Such an impermeable membrane would be included in the pump 400 only if it was desired to implant the pump 400 in an inactive state and, at some later time, activate it so as to initiate the delivery of the pharmaceutical agent contained therein. The single stage pump 400 may also include the OFF switch features shown in FIGS. 32 and 33.

The pharmaceutical agent compartment of the pumps according to embodiments of the present invention, as noted above, may contain sufentanil, for example, and may also contain other medications. Depending upon the clinical indication, the pumps according to embodiments of the present invention may be configured for intravascular, subcutaneous, epidural, intrathecal or intraventricular use. Table 1 below details exemplary maximum expected dosages of Sufentanil for above-listed uses.

TABLE 1

| | Expected Maximum Dosage of |
|---|---|
| | Sufentanil (μg/day) |
| Intravascular | 1500 |
| Subcutaneous | 1500 |
| Epidural | 500 |
| Intrathecal | 50 |
| Intraventricular | 25 |

Table 2 below shows exemplary delivery schedules for pumps according to embodiments of the present invention having a diameter of 1.8 cm and a compartment 310 having a capacity of 200 mg, a diameter of 2.8 cm and a compartment 310 having a capacity of 500 mg and a diameter of 5.0 cm and a compartment 310 having a capacity of 2000 mg over selected delivery rates (in mg/day) ranging from 0.50 mg/day to 20.0 mg/day.

Exemplary Delivery Schedule
Months of Delivery

| | 1.8 cm diameter | 2.8 cm diameter | 5.0 cm diameter |
|---|---|---|---|
| | 200 mg capacity | 500 mg capacity | 2000 mg capacity |
| Delivery Rate | (Without dose | (With dose | (With dose |
| (mg/day) | escalation) | escalation) | escalation) |
| 0.50 | 12 | — | — |
| 0.75 | 8 | 12 | — |
| 2.00 | 3.3 | 6 | — |
| 5.00 | — | 3.3 | 12 |
| 10.0 | — | — | 6 |
| 20.0 | — | — | 3.3 |

Embodiments of the present invention may be implanted under the patient's skin in an outpatient setting. The implantation procedure may be performed with a local anesthetic and may be carried out in as little as 15-20 minutes, for example. Depending upon the implant site, a small 0.5 to 0.75 inch incision may be all that is required, which incision may later be closed with one or more STERI-STRIP® skin closure devices or sutures, for example. The thin, circular shape of the pumps according to embodiments of the present invention facilitate placement thereof in a number of locations throughout the patient's body, including the chest wall, the lower back, the arms and legs, the neck and even under the scalp, to identify a few exemplary locations. It is to be understood, however, that the above list of possible implant sites is not to be construed as limiting the locations at which the present pumps may be implanted, as those of skill in this art may recognize. Embodiments of the present invention have been presented within the context of pain management and of drugs of a potency comparable to Sufentanil. However, embodiments of the present invention may be scaled appropriately to deliver any volume of drug at any potency level.

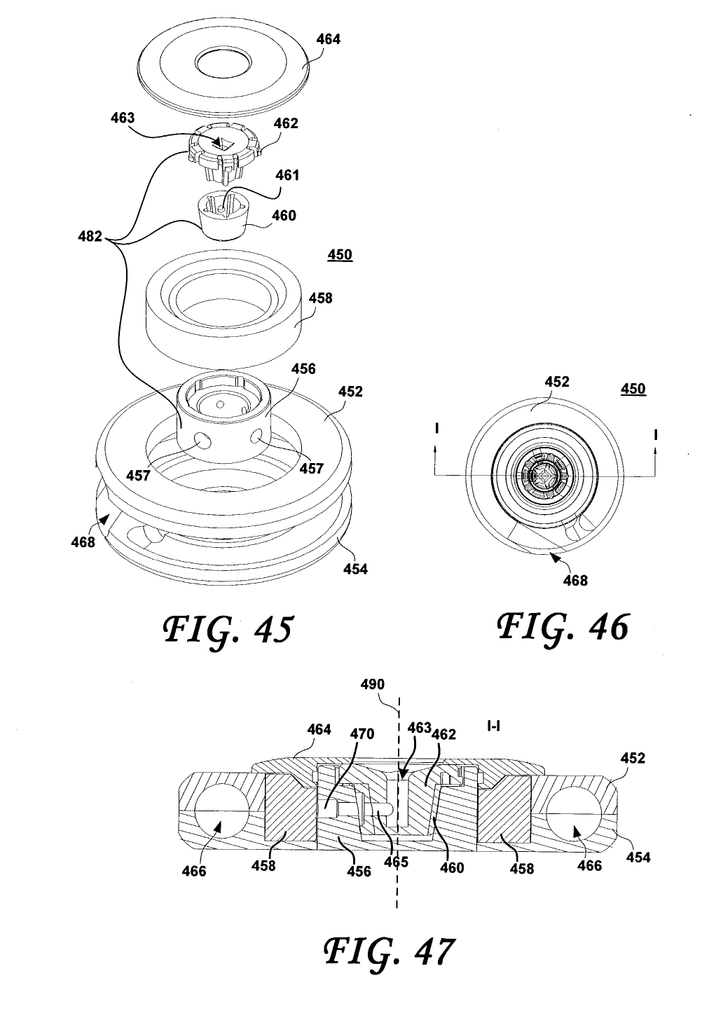

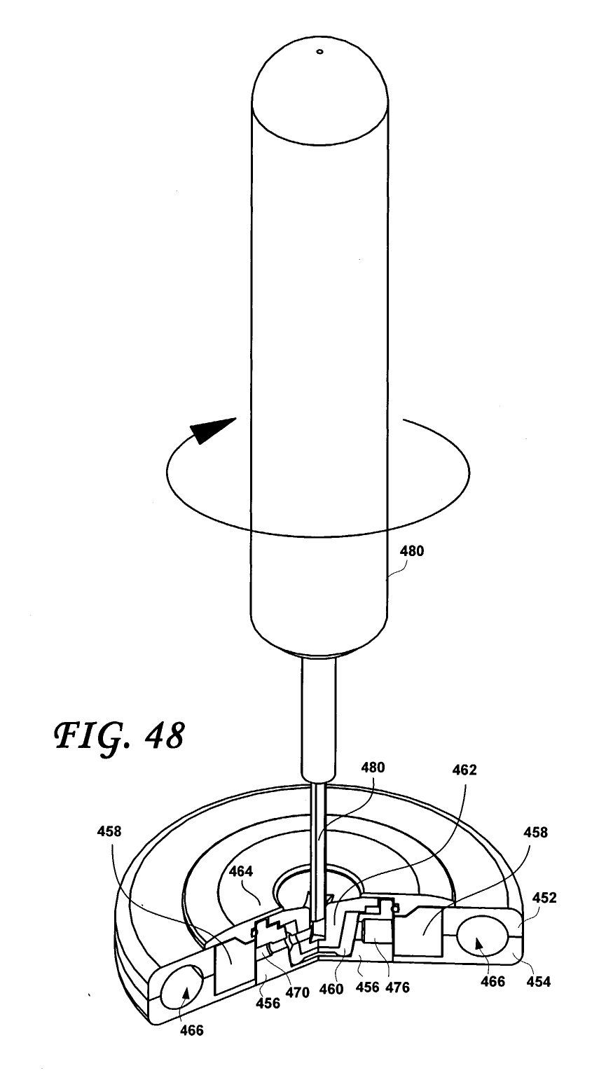

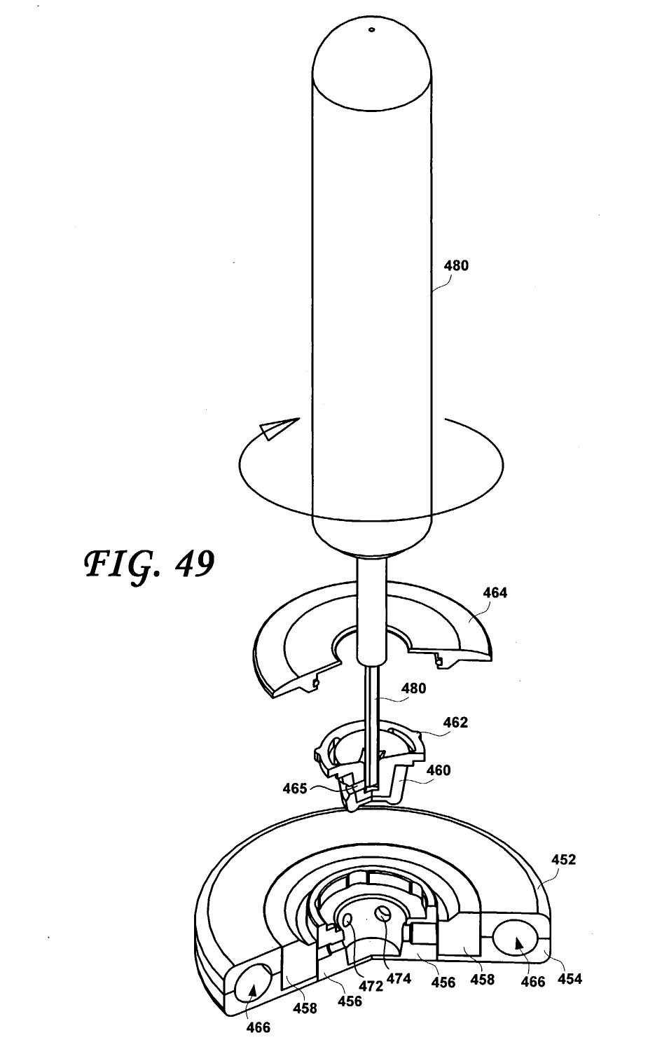

FIG. 45 shows an exploded view of the major components of an osmotic pump 450 with reversible forward and backward rate adjustability features, according to another embodiment of the present invention. FIG. 46 shows a top view of the pump 450 and FIG. 47 shows a cross sectional view of the pump 450 taken along cross-sectional line I-I. FIG. 48 shows a cutaway of pump 450 to show further structure thereof. FIG. 48 also shows the dose escalation tool 480 inserted within the pump 450. FIG. 49 shows a partially exploded view of the cutaway view of FIG. 48, revealing further interior structure of the pump 450. Considering now FIGS. 45-49 collectively, the osmotic pump 450 includes a pump housing. The pump housing may include a first housing half 452 and a second housing half 454 that, when mated to one another, define a generally toroidal-shaped pharmaceutical agent compartment 466. The pharmaceutical agent compartment 466 may contain and store one or more pharmaceutical agents. The pump 450 may include a reversible dose adjustment assembly 482 centered within the pump 450. According to this embodiment, the reversible dose adjustment assembly may include the structures referred to by numerals 456, 460 and 462, each of which is discussed in detail below. The pharmaceutical agent may be separated from the reversible dose adjustment assembly 482 and from the osmotic engine (e.g., salt block) 458 by a piston or polymeric plunger, as described in detail above. A top cover 464 seals the reversible dose adjustment assembly 482 within the pump 450, and defines an opening that exposes the top portion of reference 462.

According to the this embodiment, the fully reversible dose adjustment assembly 482 may be disposed in the center of the pump 450, replacing the membrane housing 112 described above. The dose adjustment assembly 482 of this embodiment may include an outer core 456, which includes an interior surface that defines a plurality of holes (hereafter, semipermeable membrane housings 457) that serve to house a corresponding plurality of semipermeable membranes. According to an embodiment of the present invention, the pump 450 may include four semipermeable membranes, although the present reversible dose adjustment assembly 482 may be configured for a greater or a lesser number of semipermeable membranes. Semipermeable membrane housings 470 and 476 are shown in FIG. 48, whereas FIG. 49 shows a portion of each of the semipermeable membranes housings 472 and 474. Advantageously, each semipermeable membrane that is fitted within the semipermeable membrane housings 470, 472, 474 and 476 defines a unique surface area that is configured to be exposed to both the environment of use (e.g., the patient) and exposed to the osmotic engine 458. Each semipermeable membrane may also have a unique length, which separates the osmotic engine 458 from the environment of use. It is the combination of semipermeable membrane length and surface area (among other possible semipermeable membrane characteristics (such as the composition of the semipermeable membrane(s) and combinations of characteristics), which determines the flow rate at each stage of the present multi-stage pump 450. All other membrane characteristics being equal, a smaller semipermeable membrane surface area or a longer length serves to provide a slower permeation of fluid from the environment of use to the osmotic engine 458 of the pump 450. Conversely, a larger semipermeable membrane surface area or shorter length serves to provide a faster permeation of fluid from the environment of use to the osmotic engine 458 of the pump 450. The permeation rate of fluid from the environment of use to the osmotic engine 458 is proportional to the rate at which pharmaceutical agent is delivered from the pump outlet 468 of the pump 450 to the patient. A catheter may be fitted to the outlet 468, as needed for site specific delivery, or for systemic drug delivery, the outlet 468 may be fitted with a filter assembly, such as shown at 312 in FIG. 35. Any combination of semipermeable membrane surface area and length may be used to create a desired permeation rate, and the subsequent infusion rate of the pump 450. According to the embodiment described herein, each semipermeable membrane is intended to serve as a unique pathway of permeating fluid from the environment of use to the osmotic engine 458. According to one embodiment of the present invention, only one selected semipermeable membrane allows permeation of fluid from the environment of use at any given time. According to other embodiments, a selected combination of semipermeable membranes may allow permeation of fluid from the environment of use. A seal 460 may prevent fluid from the environment of use from having access to the semipermeable membrane(s) that is/are not currently selected. The first stage of the pump (shown in cross section in FIG. 47 and in FIG. 48 at reference number 470) may have a surface area/length combination that allows the permeation of less fluid from the environment of use to the osmotic engine 458 than does the second stage, shown at reference number 472 in FIG. 49. For ease of reference herein, the stages of the pump 450 are identified by the reference numeral of the semipermeable housing that houses the currently selected semipermeable membrane. For example, the first stage 470 of the pump 450 is that stage in which the semipermeable membrane within the semipermeable membrane housing 470 allows permeation from the environment of use to the osmotic engine 458. Similarly, in the present embodiment, the second stage 472 of the pump 450 has a surface area/length combination that allows the permeation of less fluid from the environment of use to the osmotic engine 458 than does the third stage 474, shown in FIG. 49. Furthermore, in the present embodiment, the third stage 474 has a surface area/length combination that allows the permeation of less fluid from the environment of use to the osmotic engine 458 than does the fourth stage, shown at reference numeral 476 in FIG. 48. It is noted that this is but one example of the pump 450, and that other combinations of semipermeable membrane surfaces/lengths (and/or other semipermeable membrane characteristics) may be used to create different permeation rates that are selectable by an operator/user/patient. Moreover, the pump 450 need not have four stages, but may have a greater or lesser number of stages, depending upon the application.

Each semipermeable membrane may be individually selected to provide access of permeating fluid from the environment of use to the osmotic engine 458. This design allows the physician/caregiver/patient to select which semipermeable membrane is in use; thereby controlling the permeation rate and subsequently the infusion rate of the pump 450. In the embodiment shown in FIGS. 45-49, the second stage 472 has a larger surface area than the first stage 470. Therefore, selecting the second stage 472 results in a faster permeation rate and pharmaceutical agent delivery rate than would be the case had the first stage 470 been selected by the physician/caregiver. According to this embodiment of the present invention, the adjustment from one stage to another (and optionally back again, as the rate adjustment mechanism 482 is fully reversible) may be achieved by rotating the infusion rate selector 462 by a predetermined degree of rotation so that a different semipermeable membrane is selected (placed in fluid communication with the environment of use to allow permeation of the fluid from the environment of use to the osmotic pump 458). As shown, the infusion rate selector 462 may be disposed in the center of the adjustment mechanism 482. A surface of the infusion rate selector 462 defines a center conduit 465. The center conduit 465 may be generally perpendicular to the pump center axis, shown at 490 in FIG. 47. The seal 460 also defines a bore 461 that is aligned with the center conduit 465 when the rate selector 462 is mated to the seal 460. The center rotatable infusion rate selector 462 includes a surface that defines an open center pathway 463 that may be generally aligned with the center axis 490 of the pump 450 and that may make an angled turn (90 degrees, for example) to the center conduit 465. The open center pathway center 463, the center conduit 465 and the aligned bore 461 of the seal 460 together enable fluid communication from the environment of use through a selected semipermeable membrane of the pump 450. The center pathway 463 may advantageously be shaped so as to mate with a rate adjustment tool 480, shown in FIGS. 48 and 49. This center conduit 465 is the only pathway for the fluid in the environment of use to gain access to the currently selected semipermeable membrane. The center pathway 463 and the center conduit 465 together form the sole route through which permeating fluid may travel from the environment of use to the osmotic engine 458. The center conduit 465 has but one access to the semipermeable membranes, therefore, only one semipermeable membrane allows fluid permeation from the environment of use at any given time, according to one embodiment. Alternatively, the center conduit 465 may have more than one access to the semipermeable membranes, therefore, a selected combination of more than one semipermeable membrane may allow fluid permeation from the environment of use at any given time, according to another embodiment of the present invention. Since the semipermeable membranes preferably have different permeation rates, the permeation rate is therefore adjustable, since the center conduit 465 of the rotatable infusion rate selector 462 selectively provides access to each semipermeable membrane individually. In FIGS. 47, 48 and 49, the rotatable infusion rate selector 462 has been rotated such that only the semipermeable membrane with the smallest surface area (the first stage shown at reference numeral 470) has access to the environment of use. Rotating the rotatable infusion rate selector 462 by 72 degrees (assuming there are four equally spaced semipermeable membranes fitted in the semipermeable membrane housings defined within the outer core 456) by means of rate adjustment tool 480 (for example) rotates the center conduit 465 from the first stage 470 to the second stage shown at reference number 472 (see FIG. 49). The center conduit 465 now faces the semipermeable membrane of the second stage 472, which may have the 2^{nd} smallest surface area in this exemplary embodiment. Since surface area is one of the physical characteristics of the semipermeable membrane that dictate permeation rate, and since the second stage 472 may have a greater surface area than the first stage 470, then the second stage 472 may have a higher permeation rate than the first stage 470. Subsequently, with the central conduit 465 of the rotatable infusion rate selector 462 rotated to the second stage 472, the delivery rate of the pump 450 may be higher at the second stage 472 than at the first stage 470. It is the capability to select individual semipermeable membranes to vary the permeation rates across the selected semipermeable membrane that enables the pump 450 to exhibit different infusion rates of the contained pharmaceutical agent to the patient. As noted above, the rotatable infusion rate selector 462 may define more than one center conduit such as the conduit shown at reference numeral 465 and the seal 460 may define more than one bore (such as shown at 461). Having more than one center conduit would enable the physician/caregiver to select a combination of stages for an even greater permeability and thus infusion rate. Such an embodiment would give the physician/caregiver additional flexibility in selecting the ultimate infusion rate of the pump 450. After the permeability rate of the pump has been selected/changed, the physician/caregiver may retract the rate adjustment tool 480 from the center pathway 463 and retract the tool 480 from the patient and close the incision made to insert the rate adjustment tool 480 into the pump 450. This embodiment enables the physician to reversibly adjust the infusion rate of the pump 450 upward or downward long after implantation of the pump 450 into the patient by means of a small incision to allow the rate adjustment tool 480 to mate with the rotatable infusion rate selector 462 of the implanted pump 450.

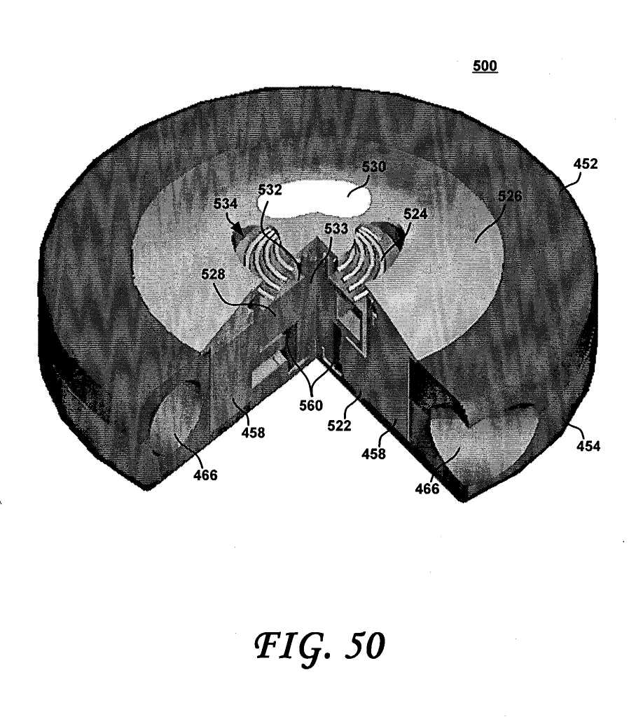

FIGS. 50-58 show aspects of another embodiment of the present invention. As opposed to the embodiment shown in FIGS. 45-49 that require the manipulation of a percutaneously inserted rate adjustment tool 480 to adjust the infusion rate of the pump 450, the embodiment of the pump 500 shown in FIGS. 50-58 includes a non-invasive, upward and downward (titratable) reversible infusion rate adjustability functionality. Indeed, the pump shown in FIGS. 50-58 is configured to perform the adjustment from one semipermeable membrane to another semipermeable membrane or from one combination of semipermeable membranes to another combination of semipermeable membranes or from one semipermeable membrane to a combination of semipermeable membranes (the pump 500 has a plurality of semipermeable membranes) using a non-invasive procedure—that is, a procedure that does not require percutaneous access in order to effectuate a change in the infusion rate of the pump 500 after implantation thereof. As described above, the semipermeable membranes may have different surface areas exposed to the osmotic engine 458. When different semipermeable membrane(s) is/are selected and exposed to the environment of use, there is a consequent change in permeation rate across the selected semipermeable membranes, and thus a change in the infusion rate of the pharmaceutical agent stored in the compartment 466 of the pump 450 into the patient.

According to an embodiment of the present invention, the semipermeable membranes may have different surface areas exposed to the osmotic engine 458. By selectively limiting access to the semipermeable membranes; that is, by covering one semipermeable membrane, several semipermeable membranes or all semipermeable membranes with a seal, such as shown at 560 in FIG. 50, the permeation rate of the fluid from the environment of use (e.g., subcutaneous or interstitial fluid) can be controlled and adjusted from zero permeation (no semipermeable membranes selected), to a first, low permeation rate (one small surface area semipermeable membrane selected) to one or more relatively higher permeation rates (one or more semipermeable membranes selected having a relatively greater surface area), and back again, if desired. As described above, each semipermeable membrane may be selectively exposed/covered individually, providing a unique permeation rate (and thus infusion rate) associated with each semipermeable membrane or with each combination of semipermeable membranes.

As shown in FIG. 50, the pump 500 may include a magnet 528, a spring member 524, an central rate adjustment module 522, a magnet sleeve 532 and one or more portals 530 defined within the top cover 526, which structures cooperate in the manner described below to enable the pump 500 to have a non-invasive and reversible dose adjustment capability. The remaining structures shown in FIG. 50 are either discussed below or may be similar to like structures shown and described above, and are referenced by the same reference numerals.

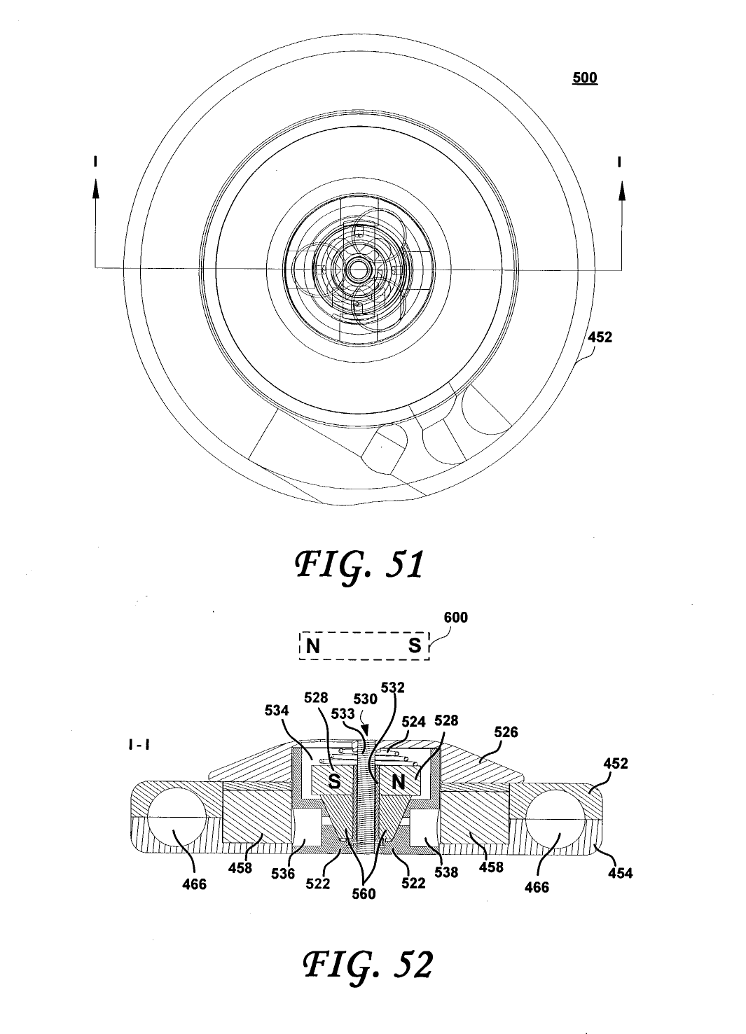

FIG. 51 is a top line drawing view of an osmotic pump with non-invasive, upward and downward reversible infusion rate adjustability, according to an embodiment of the present invention. FIG. 52 is a cross-sectional view of the osmotic pump of FIG. 51, taken along cross-sectional line I-I. Considering now FIGS. 51 and 52 collectively, the embodiment of the pump 500 shown therein may define one or more portals 530 defined in the top cover 526 of the pump 500. The portal(s) 530 enable fluid from the environment of use (e.g., the patient) to enter the pump 500. The portal(s) 530 may advantageously be covered or filled with a porous polymeric material (e.g., Gore-tex, Porex, Mupor, porous polyethylene, or a porous metal, ceramic, or other material). The porous material covering or filling the portal(s) 530 defined within the top 526 of the pump 500 is adapted to allow passage of the fluid from the environment of use and to inhibit or prevent infiltration, penetration, or adhesion of body tissue into or on the pump 500 and/or polymeric cover. The fluid from the environment of use passes through the porous polymeric material covering the portal(s) 530 and passes into the pump fluid chamber 534, and may gain access to one or more selected semipermeable membranes, such as shown at 536 and 538 in FIG. 52. The fluid chamber 534 may advantageously be filled with an aqueous solution during manufacture of the pump 500 to ensure removal of air from the fluid chamber 534. The fluid chamber 534 is contiguous to a membrane seal 560 that provides access for the fluid from the environment of use to the selected semipermeable membrane(s). For example, with reference to FIG. 52, the seal 560 may cover one of the semipermeable membranes 536, 538 and may expose the other of the semipermeable membranes 536, 538 to the aqueous fluid in the fluid chamber 534. The portion of the seal 560 immediately next to the exposed semipermeable membrane may have a slot, which allows communication of water from the fluid chamber 534 down to the exposed semipermeable membrane. In the embodiment of FIG. 52, the seal 560 is unitized with the magnet 528 and a magnet sleeve 532 to reversibly and non-invasively adjust the infusion rate of the pump 500. The assembly including the seal 560, the magnet 528 and the magnet sleeve 532 is referred herein below as the dose adjustment assembly. The dose adjustment assembly may be held in place by a spring member 524. The spring member 524 provides a biasing force configured to insure that the semipermeable membrane(s) that is/are covered by the seal 560 is/are sealed from aqueous solution in the fluid chamber 534. For example, the magnetic poles of the magnet 528 may be oriented such as shown in FIG. 52, where N and S designate the North and South poles, respectively, of the magnet 528. According to an embodiment of the present invention, the infusion rate of the pump 500 may be adjusted up or down in a non-invasive manner by coupling the magnet 528 with a strong magnetic field (provided by another magnet, such as shown in dashed lines at 600 in FIG. 52) that is external to the pump 500. Indeed, when coupled with a strong external magnet placed on the patient's skin above the pump 500, the coupled magnets 528, 600 provide the force required to overcome the biasing force of the spring member 524, to lift the dose adjustment assembly and to rotate the dose adjustment assembly to cause the seal 560 to expose another semipermeable membrane or another combination of semipermeable membranes to the aqueous solution in the fluid chamber 534.

Indeed, when a strong magnet 600 is placed on (or over) the skin overlying the implanted pump 500, and the poles of the external magnet 600 are aligned (about) 180° opposite of the pump's magnet 528 (i.e., South to North, and North to South), the magnets 600, 528 couple (are attracted) to one another. The pump magnet 528, under the influence of the external magnetic force generated by the external magnet 600, will be attracted to the external magnet 600, and the spring member 524 will compress, as it is confined in the space between the top cover 526 and the magnet 528. The attractive force of the external magnet 600 pulls the dose adjustment assembly and its seal 560 away from the pump's central rate adjustment module 522. The rate adjustment assembly slides on the magnet sleeve 532, toward the external magnet 560. Once the seal 560 is moved away from the central rate adjustment module 522, the rate adjustment assembly is free to rotate about a center post 533 in response to any rotational forces applied to the external magnet 600 by the physician or caregiver. The external magnet 600 may be rotated a predetermined angle to correspondingly rotate the rate adjustment assembly by the same predetermined angle. This predetermined angle corresponds to the angle of separation from one semipermeable membrane to another. If the pump 500 of FIG. 52 only includes the two semipermeable membranes 536, 538, the angle required to rotate the rate adjustment assembly from one of the semipermeable membranes 536, 538 to the other one of the semipermeable membranes 536, 538 is 180°, assuming that the semipermeable membranes 536, 536 are disposed diametrically apart. Likewise, if an embodiment of the pump of the present invention includes five semipermeable membranes, the angle required to rotate the rate adjustment assembly from one semipermeable membrane to the next adjacent (nearest) semipermeable membrane would be about 360°/5 or 72°, providing that the five semipermeable membranes are equally spaced around the circumference of the central rate adjustment module 522. By way of example, FIGS. 51 and 52 show a pump 500 with four semipermeable membranes. Each semipermeable membrane fitted within the central rate adjustment module 522 may have a larger or smaller surface area exposed to the osmotic engine, resulting in a higher or lower permeation rate (and hence a higher infusion rate of the pump), all other semipermeable membrane characteristics being equal. After coupling the two magnets 600, 528 and imposing a rotation on the external magnet 600 of a desired angle, the external magnet 600 is lifted straight up away from the patient's skin and away from the rate adjustment assembly, thereby de-coupling the external magnet 600 from the magnet 528 of the pump 500. Once the two magnets 600, 528 are de-coupled, the spring member 524 forces the rate adjustment assembly and its seal 560 back against the central rate adjustment module 522. The procedure described above allows a user (physician, caregiver) to adjust the infusion rate of an embodiment of the implanted osmotic pumps described herein without breaching the patient's skin (i.e., non-invasively). The pumps described herein may be designed in many different forms, with many different combinations of semipermeable membrane surface areas, using either one membrane or a plurality of membranes.

The diameters of the semipermeable membranes fitted within the central rate adjustment module 522 may be the same or may be different from one semipermeable membrane to the next. The diameter of the ends of each semipermeable membrane exposed to the environment of use may be the same as the diameter of the ends of each semipermeable membrane exposed to the osmotic engine 528. Having the same diameter typically produces equal surface areas. It may be desirable that the semipermeable membranes fitted within the central rate adjustment module 522 have different surface areas exposed to the osmotic engine 528, which would result in different permeation rates from one semipermeable membrane to another. One method of adjusting the surface area of a semipermeable membrane that is exposed to the osmotic engine 528 is to modify the diameter of the end thereof that is exposed to the osmotic engine 528. Alternatively, the end of the semipermeable membrane exposed to the osmotic engine 528 may have a diameter that is equal to the diameter of the opposite end thereof (i.e., the end exposed to the environment of use) and still have a larger surface area. Indeed, the end of the semipermeable membrane that is exposed to the osmotic engine 528 may have a modified geometry that would effectively increase the surface area of the semipermeable membrane. The surface area of the end of the semipermeable membrane adjacent to the osmotic engine 528 may be adjusted (increased) by making the end of the semipermeable membrane protrude into the osmotic engine 528 (e.g., by making the end of the semipermeable membrane that is exposed to the osmotic engine have a shape resembling a cone, ball, cylinder, etc. Alternatively, the end of the semipermeable membrane(s) exposed to the osmotic engine 458 may have a folded, convoluted or rippled surface to further increase the effective surface area without increasing the diameter thereof. The geometries of the ends of the semipermeable membranes that are exposed to the osmotic engine 528 may be selected at will to achieve the desired exposed surface area and thus achieve a desired infusion rate. Indeed, the surface area, thickness, composition and permeation rate may be freely modified to produce semipermeable membranes that result in higher infusion rates.

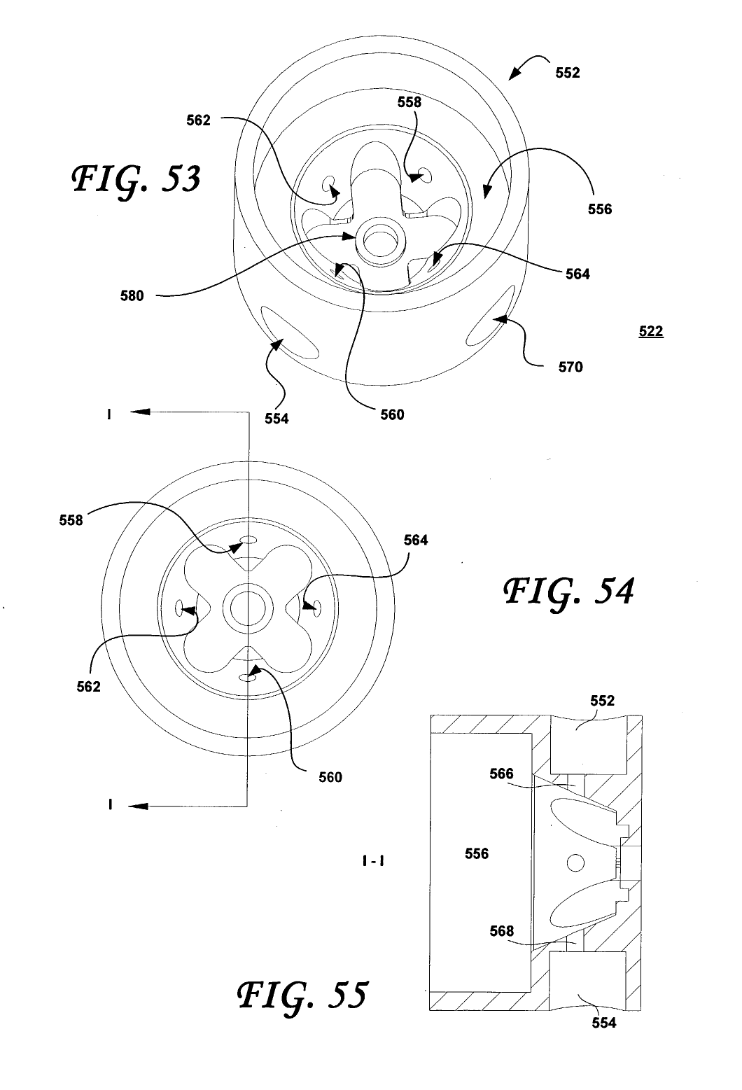

FIG. 53 is an isometric view of an exemplary central rate adjustment module 522 of an implantable osmotic pump with upward and downward reversible infusion rate adjustability, according to an embodiment of the present invention. FIG. 54 is a top view of the central rate adjustment module 522 of FIG. 53 and FIG. 55 is a cross-sectional view of the central rate adjustment module 522, taken along cross-sectional line I-I thereof. As shown in FIGS. 53-55, the central rate adjustment module 522 may be generally cylindrical and may define (preferably equally) spaced semipermeable membrane housing along the outer surface thereof. Two such semipermeable membrane housings are shown at reference numerals 552 and 554 in the cross-sectional view of FIG. 55. These semipermeable membrane housings are configured to enable the semipermeable membranes fitted therein to abut or be in fluid communication with the osmotic engine 528. When the top cover 526 is fitted to the pump 500, the interior space 556 defined by the internal surfaces of the central rate adjustment module 522 forms the fluid chamber 534. The embodiment of the central rate adjustment module 522 shown in FIGS. 53-55 is configured for four semipermeable membranes, each of which is configured to communicate with the fluid chamber 534 unless covered by the seal 560. The seal 560 has one or more openings defined therein to enable fluid from the fluid chamber 534 to reach one or more of the internal openings 558, 560, 562 or 564. One such opening is shown at 461 in the seal 460 of FIG. 45. The seal 560 and the central rate adjustment module 522 are each configured to enable the seal 560 to fit within the space 556 inside the central rate adjustment module 522.

As shown, the exemplary central rate adjustment module 522 includes an internal surface that defines four internal openings 558, 560, 562 and 564. Each of these internal openings communicates with a corresponding semipermeable membrane housing (of which only semipermeable membrane housings 552, 554 and 570 are shown in FIGS. 53 and 55). Between each internal opening and each corresponding semipermeable membrane housing of the central rate adjustment module 522 is a passageway defined within the central rate adjustment module 522. Two such passageways 566 and 568 are shown between the internal opening 558 and the semipermeable membrane housing 552 and between the internal opening 560 and the external opening 554, respectively. These passageways enable fluid from the environment of use that has entered into the fluid compartment 534 to reach the semipermeable membrane(s) fitted within the semipermeable membrane housings, unless sealed therefrom by the seal 560.

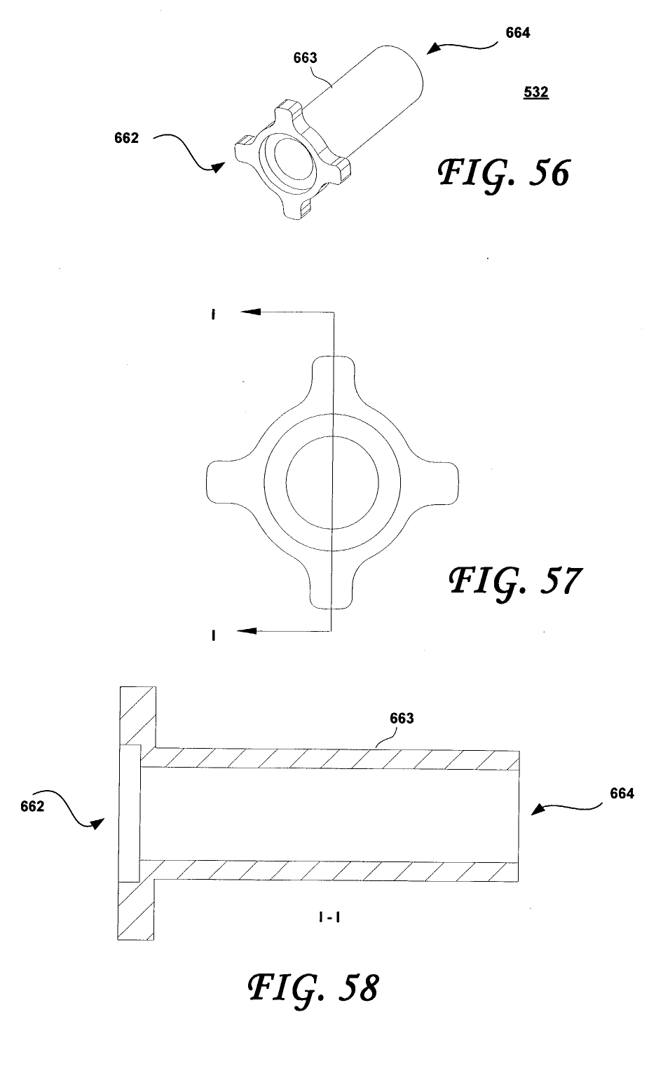

FIG. 56 is an isometric view of a magnet sleeve 532 of an implantable osmotic pump with non-invasive, upward and downward reversible infusion rate adjustability, according to an embodiment of the present invention. FIG. 57 is a plan view of the magnet sleeve 532 of FIG. 56 and FIG. 58 is a cross-sectional view of the magnet sleeve 532 of FIG. 57, taken along cross-sectional line I-I thereof. Considering now FIGS. 56-58 collectively, the magnet sleeve 532 defines a first end 662 that is configured to mate with a corresponding structure 580 within the central rate adjustment module 522. The first end 662 and the second end 664 are separated from one another by the sleeve shaft 663 to which the magnet 528 and the seal 560 are attached. The first end 662 of the magnet sleeve 532 may be keyed to the structure 580 within the central rate adjustment module 522 such that after being lifted and rotate under the influence of the external magnet 600, the magnet sleeve will only settle back within the central rate adjustment module 522 at one of a plurality of predetermined orientations that allow the permeation of fluid from the fluid chamber 534 through one of the semipermeable membranes. Also, the keying of the magnet sleeve 532 to the structure 580 keeps extraneous magnetic fields from inadvertently rotating the magnet of the dose adjustment assembly.

While the foregoing detailed description has described preferred embodiments of the present invention, it is to be understood that the above description is illustrative only and not limiting of the disclosed invention. Those of skill in this art will recognize other alternative embodiments and all such embodiments are deemed to fall within the scope of the claimed invention. Thus, the present inventions should be limited only by the claims as set forth below.

What is claimed is:

1. A pump for delivering a pharmaceutical agent, comprising:
a pump engine;
a piston;
a pharmaceutical agent compartment configured to enclose a volume of pharmaceutical agent and the piston, the pharmaceutical agent compartment being configured such that when the piston is acted upon by the pump engine, the piston moves within the pharmaceutical agent compartment and delivers the pharmaceutical agent, and
a rate adjustment assembly configured to enable a selective and reversible increase or decrease of a delivery rate of the pharmaceutical agent.

2. The pump of claim 1, wherein the rate adjustment assembly is configured to selectively vary the delivery rate of the pharmaceutical agent by percutaneous insertion and manipulation of a rate adjustment tool in the rate adjustment assembly.

3. The pump of claim 1, wherein the rate adjustment assembly is configured to vary the delivery rate of the pharmaceutical agent non-invasively when the pump is implanted into a patient.

4. The pump of claim 1, wherein the rate adjustment module is configured to enable the delivery rate of the pharmaceutical agent to be changed by application of an external magnetic field to the pump.

5. The pump of claim 1, wherein the pharmaceutical agent compartment is preloaded with the volume of the pharmaceutical agent.

6. A method of delivering a pharmaceutical agent, comprising steps of:
implanting a pump into the patient, the pump including a pump engine, a piston, a pharmaceutical agent compartment configured to enclose a volume of pharmaceutical agent and the piston, the pharmaceutical agent compartment being configured such that when the piston is acted upon by the pump engine, the piston moves within the pharmaceutical agent compartment and delivers the pharmaceutical agent, and a rate adjustment assembly configured to enable a selective and reversible increase or decrease of a delivery rate of the pharmaceutical agent;
manipulating the rate adjustment assembly to selectively increase or decrease the delivery rate of the pharmaceutical agent.

7. The method of claim 6, wherein the implanting step includes a step of making an incision in the patient near a desired implantation site and wherein the manipulating step is carried out after the implantation step and after the incision is closed.

8. The method of claim 7, wherein the manipulation step includes a step of percutaneously inserting a rate adjustment tool into the rate adjustment assembly.

9. The method of claim 7, wherein the manipulation step is carried out without breaching the patient's skin.

10. The method of claim 7, wherein the manipulation step includes a step of applying an external magnetic field near the implantation site.

11. The method of claim 10, wherein the external magnetic field applying step includes a step of rotating the external magnetic field by a selected degree of rotation.

12. An osmotic pump, comprising:
an osmotic engine;
a pump housing enclosing the osmotic engine and defining a space adapted to contain a volume of pharmaceutical agent, and
a rate adjustment module configured to enable a selective and reversible increase or decrease of a delivery rate of the pharmaceutical agent.

13. The osmotic pump of claim 12, wherein the space is preloaded with the volume of the pharmaceutical agent.

14. An osmotic pump for delivery a pharmaceutical agent, comprising:
an osmotic engine;
a pharmaceutical agent compartment adapted to contain a volume of the pharmaceutical agent;
a plurality of semipermeable membranes, one end of each of which being in communication with the osmotic engine, each of the plurality of semipermeable membranes being configured to enable an osmotic pressure differential to develop when another end thereof is selectively exposed to fluid from an environment of use, and
a rate adjustment assembly configured to selectively expose or cover at least one of the plurality of semipermeable membranes to the environment of use to selectively and reversibly increase or decrease a rate at which the pharmaceutical agent is delivered from the osmotic pump.

15. The osmotic pump of claim 14, wherein the rate adjustment module is configured to enable the selective and reversible increase or decrease of the delivery rate without direct physical contact with the pump.

16. The osmotic pump of claim 14, wherein the rate adjustment module is configured to enable the selective and reversible increase or decrease of the delivery rate through an application of an external magnetic field to the osmotic pump.

17. The osmotic pump of claim 14, wherein the rate adjustment assembly is further configured to mate with a rate adjustment tool.

18. The osmotic pump of claim 14, wherein the pharmaceutical agent compartment is preloaded with the volume of the pharmaceutical agent.

19. A method of non-invasively increasing or decreasing a dose of pharmaceutical agent delivered to a patient by a previously implanted osmotic pump, comprising the steps of:
providing a magnet;
positioning the provided magnet on or close to a skin of the patient over the previously implanted osmotic pump, and
rotating the positioned magnet by a predetermined degree of rotation, whereby the implanted osmotic pump responds to the rotating magnet by increasing or decreasing the dose of pharmaceutical agent delivered to the patient.
